(19) United States
(12) Patent Application Publication
(10) Pub. No.: US 2026/0334781 A1
(43) Pub. Date: Oct. 29, 2026

(54) AUTOMATED RHEXIS EDGE GUIDANCE FOR SURGICAL PROCEDURES

(71) Applicant: Alcon Inc., Fribourg (CH)

(72) Inventors: Zhuoran WU, Fort Worth, TX (US); Lu YIN, Fort Worth, TX (US); Vignesh SURESH, Fort Worth, TX (US); Aakash NAGARAJAN, Fort Worth, TX (US); Nikhil SCARIA, Fort Worth, TX (US); Sinchan BHATTACHARYA, Fort Worth, TX (US)

(21) Appl. No.: 19/658,103

(22) Filed: Apr. 24, 2026

Related U.S. Application Data
(60) Provisional application No. 63/795,003, filed on Apr. 25, 2025.

(51) Int. Cl. A61B 3/13; A61B 3/00; A61B 3/11; A61B 3/14

(52) U.S. Cl. CPC A61B 3/13; A61B 3/005; A61B 3/112; A61B 3/145

(57) ABSTRACT

Certain embodiments provide an optical imaging system including an ophthalmic microscope and an image processing system. The image processing system includes a camera attached to the ophthalmic microscope, a display, and a processing system. The processing system includes a processor that is configured to receive a video stream from the camera. The video stream includes a sequence of video frames, and each video frame includes image data of an eye of a patient. For each phacoemulsification video frame, the processor is configured to select a prior pupil contour C_{f} that matches a current pupil contour Cp, calculate a spatial transform based on the pupil contours C_{f} and C_{g}, apply the spatial transformation to the rhexis edge stored with the selected pupil contour C_{f} to generate a transformed rhexis edge, and generate an overlay identifying the transformed rhexis edge, and present the overlay with the image data on the display.

### CROSS-REFERENCE TO RELATED APPLICATIONS

This application claims the benefit of U.S. Provisional Application Ser. No. 63/795,003 (filed on Apr. 25, 2025), the content of which is incorporated by reference herein in its entirety.

### BACKGROUND

A variety of diseases or conditions associated with an eye may be treated through ophthalmic surgical procedures. Examples of ophthalmic surgical procedures include vitreoretinal surgery, cataract surgery, glaucoma surgery, laser eye surgery (e.g., Laser-Assisted In Situ Keratomileusis or LASIK), and the like. For example, vitreoretinal surgery is a type of eye surgery that treats problems with the retina or the vitreous and may be performed for treating conditions such as diabetic traction retinal detachment, diabetic vitreous hemorrhage, macular hole, retinal detachment, epimacular membrane, and many other ophthalmic conditions. As another example, cataract surgery is a type of eye surgery that replaces a clouded natural lens in the eye of a patient with an artificial intraocular lens (IOL) to restore clear vision.

### BRIEF DESCRIPTION OF THE DRAWINGS

FIG. 1 depicts a cross-sectional diagram of an eye of a patient during an ophthalmic surgery, in accordance with certain embodiments of the present disclosure.

FIG. 2A depicts an example optical imaging system, in accordance with certain embodiments of the present disclosure.

FIG. 2B presents a diagram of the example optical imaging system depicted in FIG. 2A, in accordance with certain embodiments of the present disclosure.

FIG. 2C depicts a side view of the example stereoscopic microscope depicted in FIG. 2A, in accordance with certain embodiments of the present disclosure.

FIG. 3A depicts another example optical imaging system, in accordance with certain embodiments of the present disclosure.

FIG. 3B presents a diagram of the example optical imaging system depicted in FIG. 3A, in accordance with certain embodiments of the present disclosure.

FIG. 3C depicts a left side view of the example monoscopic microscope depicted in FIG. 3A, in accordance with certain embodiments of the present disclosure.

FIG. 4 depicts an example data flow diagram that illustrates the steps of the method for registering and visualizing a rhexis edge that are performed during the capsulorhexis phase, in accordance with certain embodiments of the present disclosure.

FIGS. 5A, 5B depict example data flow diagrams that illustrate the steps of the method for registering and visualizing a rhexis edge that are performed during the phacoemulsification phase, in accordance with certain embodiments of the present disclosure.

FIG. 6A depicts image data from a video frame with an example preliminary overlay, in accordance with certain embodiments of the present disclosure.

FIG. 6B depicts the example preliminary overlay of FIG. 6A, in accordance with certain embodiments of the present disclosure.

FIG. 7A depicts the image data from a video frame with an example overlay, in accordance with certain embodiments of the present disclosure.

FIG. 7B depicts the example overlay of FIG. 7A, in accordance with certain embodiments of the present disclosure.

### DETAILED DESCRIPTION

Embodiments of the present disclosure will now be described with reference to the drawing figures, in which like reference numerals refer to like parts throughout.

Cataract surgery typically begins with the creation of a continuous curvilinear capsulorhexis (CCC), which involves forming a smooth, continuous, circular opening in the anterior capsule of the crystalline lens. Guided by a magnified and illuminated view of the eye provided by an ophthalmic microscope of an optical imaging system, the surgeon creates a self-sealing main incision (such as 2 to 3 mm) in the cornea and one or more self-sealing side-port incisions (such as 1 to 1.5 mm). Using a cystotome needle or capsulorhexis forceps, the surgeon initiates a central puncture in the anterior capsule and then tears the capsular tissue in a controlled curvilinear manner using the capsulorhexis forceps to produce a well-centered opening, typically 5 to 6 mm in diameter. The resulting CCC provides a strong, elastic rim known as the capsulorhexis edge (referred to herein as the rhexis edge) with high tear resistance. The CCC provides the foundation for subsequent surgical maneuvers by allowing safe access to the lens nucleus while preserving the integrity of the capsular bag for IOL support.

Following capsulorhexis, phacoemulsification is performed to remove the opacified lens material. After hydrodissection and hydrodelineation separate the nucleus from the cortex and capsule, an ultrasonic phacoemulsification handpiece is introduced through the main corneal incision. The probe delivers high-frequency ultrasonic energy (typically around 40,000 Hz) combined with irrigation and aspiration to fragment, emulsify, and simultaneously aspirate the cataractous nucleus into small pieces that are removed from the eye. Various nuclear disassembly techniques, such as divide-and-conquer or chopping methods, may be employed depending on lens density. Once the nucleus and epinucleus are removed, residual cortical material is meticulously aspirated using irrigation/aspiration instruments, leaving an empty capsular bag with an intact posterior capsule. This step minimizes incision size, reduces surgical trauma, and enables rapid visual rehabilitation compared to older extracapsular techniques.

The IOL is then inserted to restore refractive power. A foldable IOL, typically made of acrylic or silicone, is loaded into an injector system and implanted through the main corneal incision under viscoelastic cover. The IOL is carefully positioned and centered within the capsular bag so that the rhexis edge overlaps the IOL optic for stable fixation and to reduce the risk of posterior capsule opacification or decentration. After proper placement, the viscoelastic material is thoroughly removed via irrigation and aspiration to prevent postoperative pressure spikes, and the incisions are hydrated or sutured if necessary for watertight closure. This implantation completes the replacement of the natural lens, providing immediate optical correction and long-term visual stability.

### Technical Problem

Existing ophthalmic surgical visualization systems are unable to maintain accurate spatial awareness of the rhexis edge after completion of the capsulorhexis phase of a cataract surgery. While the rhexis edge is visible during the capsulorhexis phase, the rhexis edge may not be visible during the phacoemulsification phase or the IOL implantation and alignment phase. For example, the rhexis edge may become obscured due to changes in illumination, occlusion by surgical instruments, and deformation of ocular tissue during the phacoemulsification phase. As a result, real-time intraoperative imaging fails to provide sufficient information to localize the previously-formed incision geometry of the rhexis edge during the phacoemulsification phase.

This deficiency in image-based visualization systems leads to a loss of spatial registration between prior and current surgical phases, which increases the risk of unintended and adverse interaction with the anterior lens capsule. Conventional image display or augmentation techniques fail to provide a technically reliable mechanism for persistently tracking and re-registering the rhexis edge across temporally-separated video frames in the presence of anatomical variability and intraoperative motion.

### Technical Solution

Embodiments of the present disclosure advantageously provide improved image processing and visualization systems that detect the rhexis edge that is formed by the surgeon during the capsulorhexis phase, and then generate and display an overlay that identifies the detected rhexis edge during a subsequent phase, such as the phacoemulsification phase. For example, identifying the rhexis edge in the overlay may prevent unintended and adverse interaction with the anterior lens capsule, such as inadvertently grabbing the lens capsule during the phacoemulsification phase.

In certain embodiments, a preliminary overlay that identifies the detected rhexis edge may be generated and displayed during the capsulorhexis phase. Additionally, certain anatomical features of the eye that are detected during the capsulorhexis and phacoemulsification phases may be identified in the respective overlays, such as the pupil boundary, the limbus boundary, and so on.

In certain embodiments, one or more rhexis guidance circles may be generated and displayed in the preliminary overlay during the capsulorhexis phase to assist the surgeon during the creation of the CCC.

In certain embodiments, the path of the tip of the forceps used by the surgeon to create the CCC during the capsulorhexis phase may be tracked and identified in the preliminary overlay. Additional guidance features may also be generated and identified in the preliminary overlay, such as deviation vectors, live correction arrows, and so on. The additional guidance features may also include non-visual cues, such as haptic cues, audio cues, and so on.

In certain embodiments, the boundary of the lens capsule may be determined based on pre-operative biometry and identified in the overlay during the phacoemulsification phase. A visual, haptic, or audio proximity alert may warn the surgeon that a phacoemulsification instrument tip has approached within a predetermined distance of the lens capsule boundary.

In certain embodiments, certain pupil characteristics may be determined based on the rhexis edge which may indicate to the surgeon that one or more preventative measures should be taken, such as implantation of a capsular tension ring after capsulorhexis, before or during phacoemulsification, and so on.

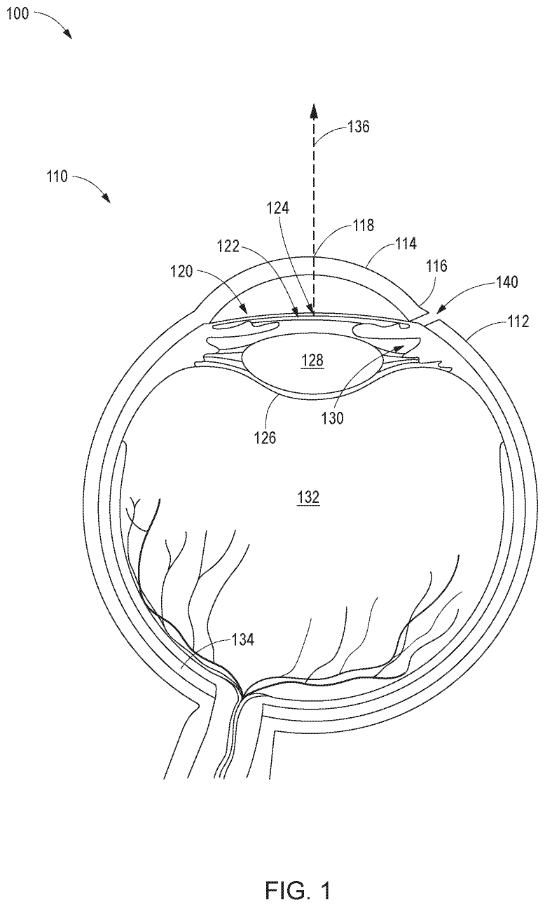

FIG. 1 depicts a cross-sectional diagram 100 of an eye 110 of a patient during an ophthalmic surgery, in accordance with certain embodiments of the present disclosure.

Various anatomical structures of the eye 110 are depicted, including the sclera 112, the cornea 114, the limbus 116 (the junction between the sclera 112 and the cornea 114), the corneal apex 118, the iris 120, the pupil 122, the pupil center 124, the lens capsule 126, the crystalline lens 128, the ciliary body 130, the posterior chamber 132 containing the vitreous humor, and the retina 134. The corneal apex 118 represents the most anterior point of the cornea 114 and typically corresponds to the region of greatest curvature (steepest portion) of the anterior corneal surface. The main corneal incision 140 for the CCC procedure described above is also illustrated.

The visual axis 136 of the eye 110 is illustrated as a reference line passing through the corneal apex 118 and the pupil center 124. This axis approximates the path of light rays that achieve optimal visual acuity when the eye fixates on a distant object, playing a critical role in determining the eye's precise focusing and alignment for high-resolution central vision during fixation tasks.

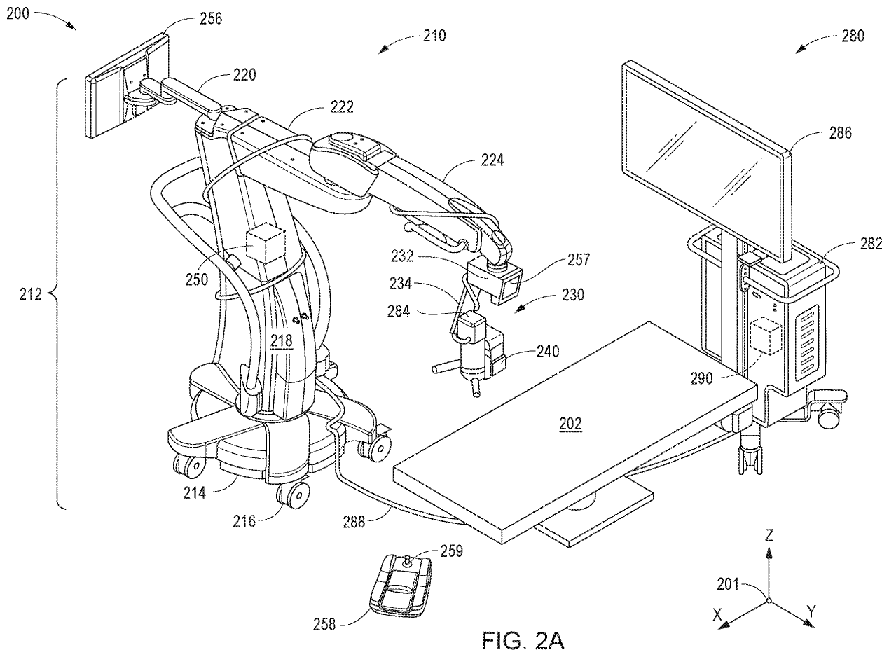

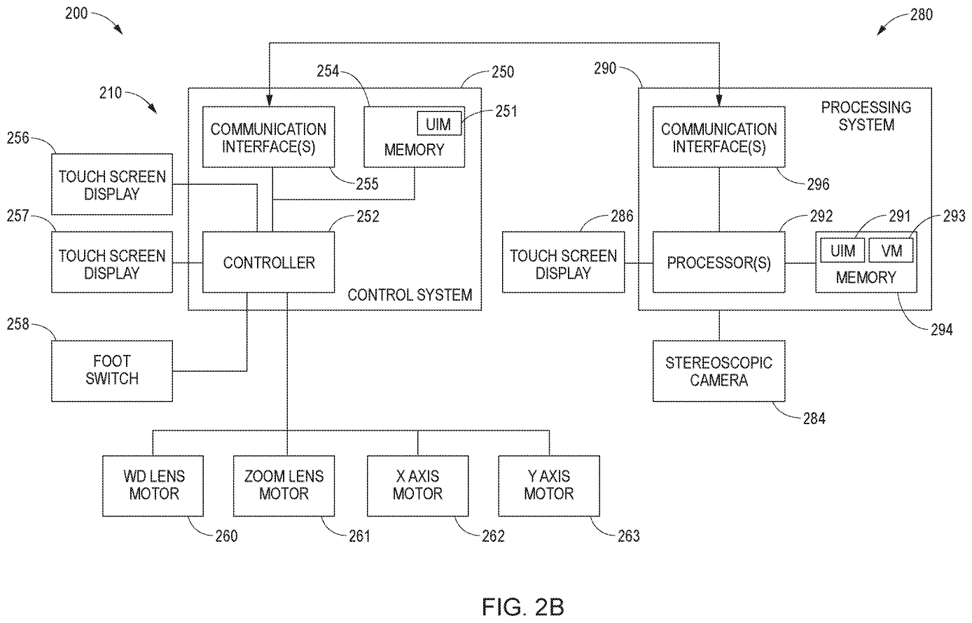

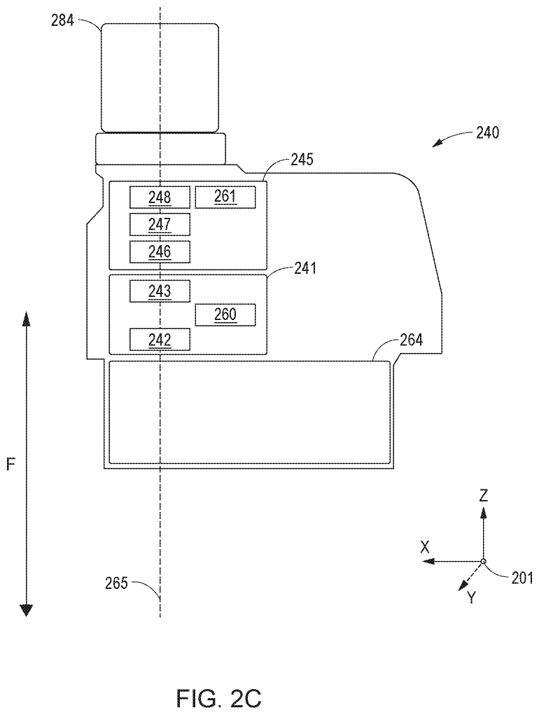

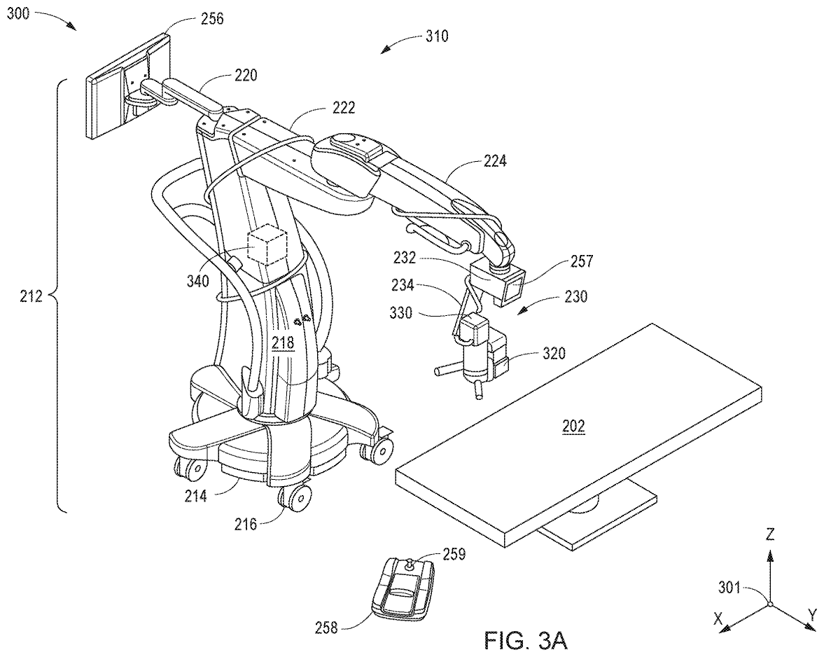

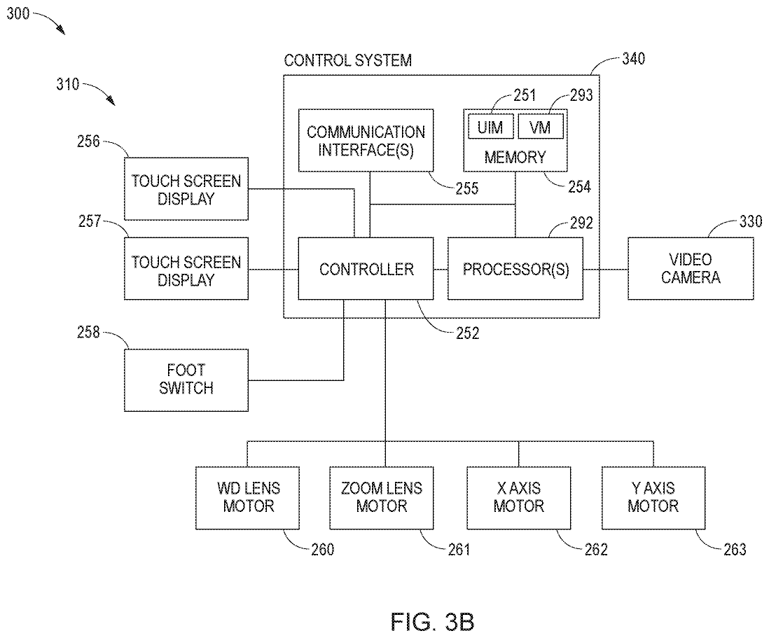

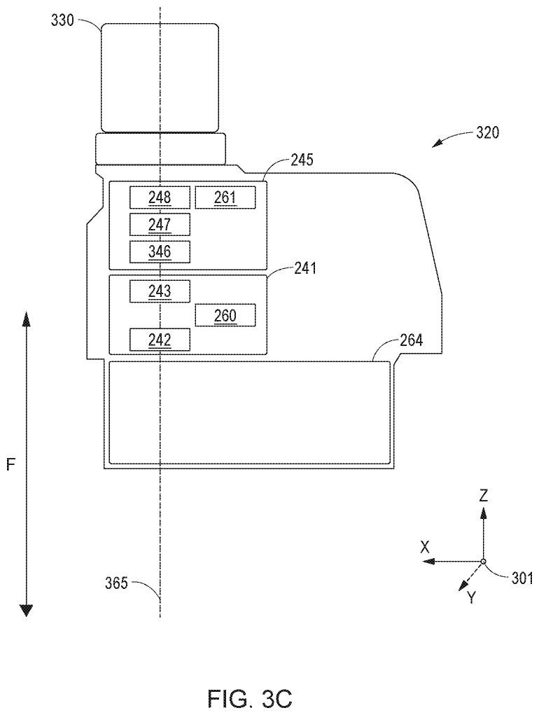

Examples of optical imaging systems are described below with respect to FIGS. 2A, 2B, 2C, 3A, 3B, and 3C.

FIG. 2A depicts an optical imaging system 200, in accordance with certain embodiments of the present disclosure. An orthogonal coordinate system 201 is depicted and includes X, Y, and Z axes.

In certain embodiments, the optical imaging system 200 includes an ophthalmic microscope 210 and an ophthalmic surgical image processing system 280 located proximate to the patient bed 202.

The ophthalmic microscope 210 includes a floor stand 212 and a microscope optical head 230. The floor stand 212 includes, among other things, a support base 214 having a set of wheels 216 with brakes, a support pillar 218 that is rotatably coupled to the support base 214, a support arm 222 that is attached to the support pillar 218, and an articulated arm 224 that is rotatably coupled to the support arm 222. The articulated arm 224 may be raised and lowered to place the microscope optical head 230 at a preferred height over the head of a patient disposed on the patient bed 202.

The microscope optical head 230 includes a base unit 232 that is rotatably coupled to the end of the articulated arm 224, a yoke 234 that is coupled to the base unit, a stereoscopic microscope 240 that is rotatably coupled to the yoke 234, and a stereoscopic camera 284 attached to the stereoscopic microscope 240. The base unit 232 includes an X axis motor and a Y axis motor that are configured to move the stereoscopic microscope 240 in the XY plane. In some embodiments, the base unit 232 also includes a Z axis motor that is configured to move the stereoscopic microscope 240 in the Z direction.

A control system 250 is located within the floor stand 212 (such as within the support pillar 218) and is configured to control the operation of the microscope optical head 230. The control system 250 may control certain functions of the stereoscopic microscope 240 (such as the focus, the magnification, the illumination, the inverter, and so on), the position of the stereoscopic microscope 240 in two- or three-dimensional space (such as orthogonal X, Y directions, orthogonal X, Y, Z directions, and so on).

A touch screen display 256 may be coupled to the control system 250 and attached to the support pillar 218 via a movable arm 220. Another touch screen display 257 may be coupled to the control system 250 and attached to the microscope optical head 230. The control system 250 generates and presents a user interface (UI) on the touch screen display 256 and another UI on the touch screen display 257. The touch screen displays 256 and 257 are configured to receive inputs from the surgeon and provide those inputs to the control system 250 to control certain functions of the stereoscopic microscope 240, the position of the stereoscopic microscope 240 in two- or three-dimensional space, and so on.

A wireless (or wired) foot switch 258 is also coupled to the control system 250 and includes a joystick 259 and control switches that may be actuated by the surgeon. Similar to the UIs, the foot switch 258 receives inputs from the surgeon and provides those inputs to the control system 250 to control certain functions of the stereoscopic microscope 240, the position of the stereoscopic microscope 240 in two- or three-dimensional space, and so on.

The ophthalmic surgical image processing system 280 includes a console 282, the stereoscopic camera 284 attached to the stereoscopic microscope 240, a touch screen display 286 attached to the console 282, a signal cable 288, and a processing system 290. The signal cable 288 couples the stereoscopic camera 284 to the processing system 290. The stereoscopic camera 284 generates a video stream that is provided to the processing system 290 for further processing. The video stream includes a sequence of video frames, and each video frame includes stereoscopic image data (also known as left and right 2D image data). The video stream may have a video frame rate of 30 fps, 60 fps, at least 30 fps, and so on. The processing system 290 may derive range data from the left and right 2D image data to generate three-dimensional stereoscopic image data (also known as 3D image data).

In certain embodiments, the processing system 290 is located within the console 282. In some embodiments, the processing system 290 may be located in the floor stand 212 (e.g., in the support pillar 218), and the console 282 and the signal cable 288 are not provided. The touch screen display 286 may be attached to the floor stand 212, a wall, a ceiling, or a separate floor stand, and may be coupled to the processing system 290 via a signal cable or a wireless communication link. Alternatively, the touch screen display 256 may be configured to replace the touch screen display 286.

The processing system 290 is configured to receive and process the video stream from the stereoscopic camera 284 and then display the processed stereoscopic image data on the touch screen display 286 (and/or the touch screen displays 256, 257). Additionally, the processing system 290 is configured to, among other things, generate an overlay that identifies a transformed rhexis edge, and present the overlay with the image on the touch screen display 286 (and/or the touch screen displays 256, 257), as described in more detail below. In some embodiments, the processing system 290 may be configured to present the overlay onto a display that is located in the optical pathway of the stereoscopic microscope 240 for viewing with the image through the binoculars.

The processing system 290 is coupled to the control system 250, via the signal cable 288 or a separate electrical or optical signal cable, and may also be configured to send certain processed data to the controller 252. In some embodiments, the signal cable 288 may be replaced by a wireless communication link between the control system 250 and the processing system 290.

FIG. 2B presents a block diagram of the optical imaging system 200, in accordance with certain embodiments of the present disclosure.

The optical imaging system 200 includes the ophthalmic microscope 210 and the ophthalmic surgical image processing system 280. The ophthalmic microscope 210 includes, among other things, the control system 250, the touch screen displays 256 and 257, the foot switch 258, an X axis motor 262, a Y axis motor 263, a working distance (WD) lens motor 260, and a zoom lens motor 261.

The control system 250 includes, among other things, a controller 252, a memory 254 coupled to the controller 252, and one or more communication interfaces 255 coupled to the controller 252. The controller 252 is coupled to the touch screen displays 256 and 257, the foot switch 258, the X axis motor 262, the Y axis motor 263, the WD lens motor 260, and the zoom lens motor 261.

The controller 252 may be a processor, a microprocessor, a microcontroller, a programmable logic controller (PLC), a field programmable gate array (FPGA), an application specific integrated circuit (ASIC), and so on.

The memory 254 may include any combination of random access memory (RAM), dynamic RAM (DRAM), static RAM (SRAM), read only memory (ROM), flash memory, cache memory, and/or any other type of non-transitory computer-readable medium. The memory 254 stores a user interface module (UIM) 251 that is executed by the controller 252.

The communication interface 255 may include one or more wired or wireless communication interfaces, such as a Universal Serial Bus (USB) interface, an Ethernet interface, a WiFi interface, a Bluetooth interface, and so on.

As described above, the controller 252 is configured to receive inputs from the surgeon, via the UIs presented on the touch screen displays 256 and/or 257 and/or the foot switch 258, to control certain functions of the stereoscopic microscope 240, the position of the stereoscopic microscope 240 in two- or three-dimensional space, and so on. For example, in response to receiving a command from an input device (e.g., the foot switch 258, and so on.), the controller 252 may command the X axis motor 262 and/or the Y axis motor 263 to move the stereoscopic microscope 240 in the XY plane.

The ophthalmic surgical image processing system 280 includes, among other things, the stereoscopic camera 284, the processing system 290, and the touch screen display 286. The processing system 290 includes, among other things, a processor 292, a memory 294 coupled to the processor 292, and one or more communication interfaces 296 coupled to the processor 292.

The processor 292 may be a microprocessor, a microcontroller, a programmable logic controller (PLC), a field programmable gate array (FPGA), an application specific integrated circuit (ASIC), and so on. In certain embodiments, the processor 292 may include one or more single-core or multi-core processors that are configured to process stereoscopic image data, execute ML processes, and so on. In other embodiments, the processor 292 may be a system-on-chip (SoC) that includes one or more single-core or multi-core processors and one or more special processors, such as a graphics processing unit (GPU), a neural processing unit (NPU), and so on, that are configured to process stereoscopic image data, execute ML processes, and so on.

Generally, the processor 292 is configured to receive and process the video stream from the stereoscopic camera 284 via the signal cable 288 and then present the processed stereoscopic image data on the touch screen display 286. Additionally, the processor 292 is configured to select the pupil contour (C_{f}) that matches the second pupil contour (C_{g}), calculate a spatial transformation based on the pupil contour (C_{f}) and the second pupil contour (C_{g}), apply the spatial transformation to the rhexis edge stored with the selected pupil contour (C_{f}) to generate a transformed rhexis edge, generate an overlay identifying the transformed rhexis edge, and present the overlay with the image on the touch screen display 286 (and/or the touch screen displays 256, 257), as described in more detail below. The processor 292 may also be configured to send certain processed data to the controller 252.

The memory 294 may include any combination of random access memory (RAM), dynamic RAM (DRAM), static RAM (SRAM), read only memory (ROM), flash memory, cache memory, and/or any other type of non-transitory computer-readable medium. The memory 294 stores a user interface module (UIM) 291 and a visualization module (VM) 293 that are executable by the processor 292. The VM 293 may be configured to process stereoscopic image data (e.g., left and right images).

The communication interface 296 may include one or more wired or wireless communication interfaces, such as a Universal Serial Bus (USB) interface, an Ethernet interface, a WiFi interface, a Bluetooth interface, and so on. The communication interface 296 is coupled to the communication interface 255 using a wired (or wireless) connection, and the processor 292 exchanges data with the controller 252 over the wired (or wireless) connection.

FIG. 2C depicts a left side view of the stereoscopic microscope 240, in accordance with certain embodiments of the present disclosure. The orthogonal coordinate system 201 is also depicted.

Generally, the stereoscopic microscope 240 includes an illumination system 264, an optical assembly 241, a zoom assembly 245, and left and right optical units, such as binoculars with left and right lenses, prisms, and eyepieces. In certain embodiments, the stereoscopic camera 284 may replace the binoculars, while in other embodiments, the stereoscopic camera 284 may be used in combination with the binoculars. The stereoscopic camera 284 provides left and right optical sensors. The stereoscopic microscope 240 has an optical axis 265.

The optical assembly 241 includes a front working distance lens 242 and a rear working distance lens 243 disposed within a housing. A focal plane is located at a distance equal to the focal length F from a principal plane of the optical assembly 241. The optical assembly 241 is configured to provide a variable working distance W, which sets a plane of the target site or scene that is in focus. The controller 252 (not shown in FIG. 2C) is configured to selectively command the WD lens motor 260 coupled to the rear working distance lens 243 (and/or the front working distance lens 242) to change the spacing between the rear working distance lens 243 and the front working distance lens 242 to adjust the focus in response to input from the surgeon, such as a focus position received from the foot switch 258 or from the UI displayed on the touch screen display 257. The optical assembly 241 is configured to provide a single view of the target site to the stereoscopic camera 284 via the left optical path or the right optical path.

The zoom assembly 245 includes zoom lenses that change a focal length and angle of view of the target site to provide zoom magnification. In certain embodiments, the zoom assembly 245 includes a front lens set 246, a zoom lens unit 247, and a lens barrel set 248. The front lens set 246 may include respective positive converging lenses (for the right and left optical paths) to direct light to respective lenses in the zoom lens unit 247. The lateral position of the front lens set 246 may define a beam from the optical assembly 241 that is propagated to the zoom lens unit 247. The front lens set 246 may include lenses with adjustable radial and axial positions.

The zoom lens unit 247 may form an afocal zoom system for changing the size of a field-of-view (e.g., a linear field-of-view) by changing the size of the light beam propagated to the lens barrel set. The zoom lens unit 247 may include a front zoom lens set with respective right and left front zoom lenses. The zoom lens unit 247 may include a rear zoom lens set with respective right and left front zoom lenses. The front zoom lens set may include positive converging lenses while the rear zoom lens set may include negative diverging lenses. The controller 252 is configured to selectively command the zoom lens motor 261 to move the lens barrel set 248 along the optical axis 265 to increase or decrease the magnification in response to input from the surgeon, such as a magnification position received from the foot switch 258 or from the UI displayed on the touch screen display 257.

FIG. 3A depicts an optical imaging system 300, in accordance with certain embodiments of the present disclosure. An orthogonal coordinate system 301 is depicted and includes X, Y, and Z axes.

In certain embodiments, the optical imaging system 300 includes an ophthalmic microscope 310, which includes many of the same components as the ophthalmic microscope 210 (which are identified using the same reference characters). Notably, the microscope optical head 230 includes a monoscopic microscope 320 that is rotatably coupled to the yoke 234, and a video camera 330 that is attached to the monoscopic microscope 320. The video camera 330 generates a video stream that is provided to the control system 340 for further processing. The video stream includes a sequence of video frames, and each video frame includes monoscopic (or 2D) image data. The video stream may have a video frame rate of 30 fps, 60 fps, at least 30 fps, and so on.

The control system 340 is located within the floor stand 212 (such as within the support pillar 218) and is configured to control the operation of the microscope optical head 230. The control system 340 may control certain functions of the monoscopic microscope 320 (such as the focus, the magnification, the illumination, the inverter, and so on), the position of the monoscopic microscope 320 in two- or three-dimensional space (such as orthogonal X, Y directions, orthogonal X, Y, Z directions, and so on).

The control system 340 is configured to receive and process the video stream from the video camera 330 and then display the processed monoscopic image data on the touch screen display 256 (and/or the touch screen display 257). Additionally, the control system 340 is configured to, among other things, generate an overlay that identifies a transformed rhexis edge, and present the overlay with the image on the touch screen display 256 (and/or touch screen display 257), as described in more detail below. In some embodiments, the control system 340 may be configured to present the overlay onto a display that is located within the optical pathway of the monoscopic microscope 320 for viewing with the image through the eyepieces.

FIG. 3B presents a block diagram of the optical imaging system 300, in accordance with certain embodiments of the present disclosure.

In certain embodiments, the optical imaging system 300 includes an ophthalmic microscope 310. The ophthalmic microscope 310 is generally the same as the ophthalmic microscope 210, and the control system 340 is generally the same as the control system 250. The common components between the ophthalmic microscope 310 and the ophthalmic microscope 210 are identified in FIG. 3B using the same reference numbers used in FIG. 2B.

The control system 340 includes the processor 292 and the VM 293 from the processing system 290 depicted in FIG. 2B. The VM 293 is stored in the memory 254 and may be configured to process monoscopic image data (e.g., single images) instead of (or in addition to) stereoscopic image data.

Generally, the processor 292 is configured to receive and process monoscopic image data from the video camera 330 and then present the processed monoscopic image data on the touch screen display 256 (and/or touch screen display 257). Additionally, the processor 292 is configured to select the pupil contour (C_{f}) that matches the second pupil contour (C_{g}), calculate a spatial transformation based on the pupil contour (C_{f}) and the second pupil contour (C_{g}), apply the spatial transformation to the rhexis edge stored with the selected pupil contour (C_{f}) to generate a transformed rhexis edge, generate an overlay identifying the transformed rhexis edge, and present the overlay with the image on the touch screen display 256 (and/or touch screen display 257), as described in more detail below. The processor 292 may also be configured to send certain processed data to the controller 252.

FIG. 3C depicts a left side view of the monoscopic microscope 320, in accordance with certain embodiments of the present disclosure. The orthogonal coordinate system 301 is also depicted.

Generally, the monoscopic microscope 320 includes an illumination system 264, a monoscopic version of the optical assembly 241, a monoscopic version of the zoom assembly 245, an optical unit, prisms, and eyepieces. The monoscopic version of the optical assembly 241 and the monoscopic version of the zoom assembly 245 function as described above using a single optical path (e.g., the left optical path). In certain embodiments, the video camera 330 replaces the eyepieces and provides an optical sensor, while in other embodiments, the video camera 330 may be used in combination with the eyepieces. The monoscopic microscope 320 has an optical axis 365.

As discussed above, while the rhexis edge is visible during the capsulorhexis phase, the rhexis edge may not be visible or may be more difficult to see during the phacoemulsification phase. Embodiments of the present disclosure advantageously “remember” the surgeon's work during the capsulorhexis phase by registering the rhexis edge along with other anatomical features, such as pupil contours, limbus contours, and so on, for use during the phacoemulsification phase (and/or the IOL implantation and alignment phase). Other anatomical features, such as sclera contours, pupil centers, and so on, as well as other data, such as optical coherence tomography (OCT) data acquired during the surgical procedure, may assist the registration and visualization of the rhexis axis. Certain embodiments provide a two-dimensional (2D) rhexis registration and visualization method that processes monoscopic image data. Other embodiments provide a combined 2D/three-dimensional (3D) rhexis registration and visualization method that is slightly different than the 2D rhexis registration and visualization method that processes stereoscopic image data.

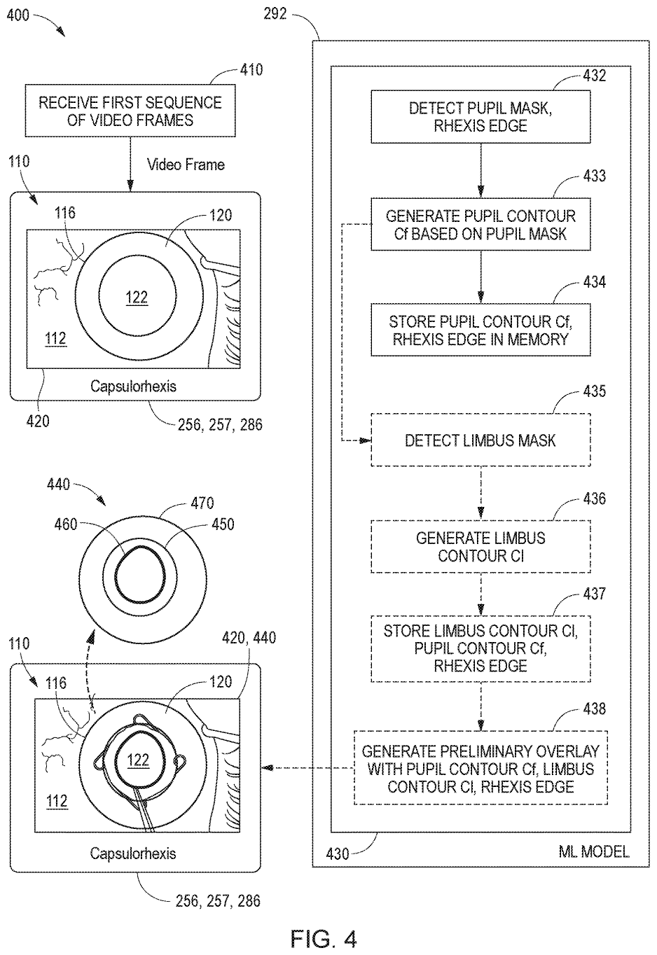

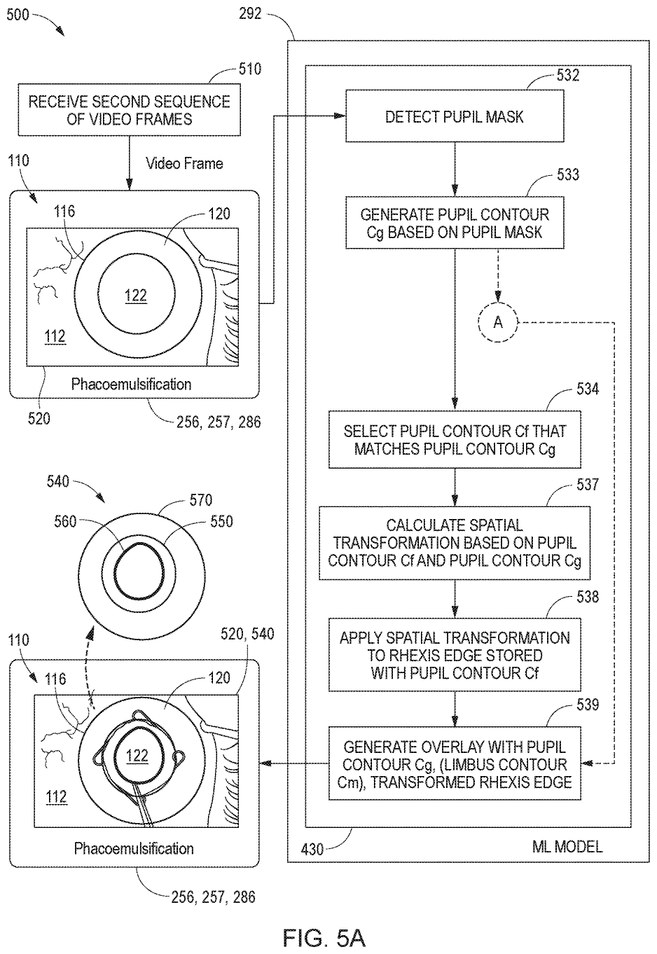

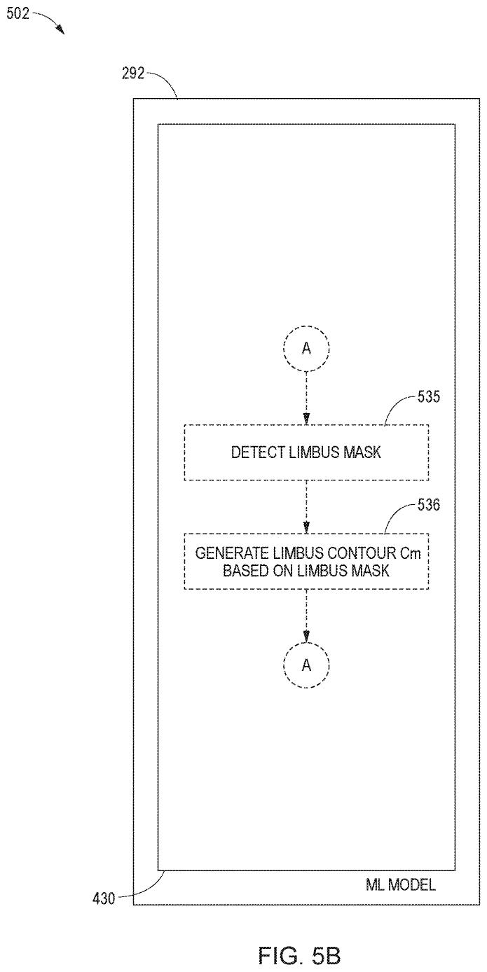

FIGS. 4, 5A, and 5B depict a method for registering and visualizing a rhexis edge during an ophthalmic surgical procedure, in accordance with embodiments of the present disclosure.

The method for registering and visualizing the rhexis edge during an ophthalmic surgical procedure is performed by an ophthalmic imaging system, such as the optical imaging systems 200, 300 described above in relation to FIGS. 2A-2C and 3A-3C (respectively).

As described above, the optical imaging system 200 includes the ophthalmic microscope 210 and the ophthalmic surgical image processing system 280. The stereoscopic camera 284 is attached to the stereoscopic microscope 240 and generates a video stream that is provided to the ophthalmic surgical image processing system 280 for further processing. The video stream includes a sequence of video frames, and each video frame includes stereoscopic image data of the eye during the ophthalmic surgery. The optical imaging system 200 performs the 2D rhexis registration and visualization method using the left image, the right image, or a combined left/right image from the stereoscopic image data, and performs the combined 2D/3D rhexis registration and visualization method using the stereoscopic image data.

Further, as described above, the optical imaging system 300 includes the ophthalmic microscope 310. The video camera 330 is attached to the monoscopic microscope 320 and generates a video stream that is provided to the control system 340 for further processing. The video stream includes a sequence of video frames, and each video frame includes monoscopic image data of the eye during the ophthalmic surgery. The optical imaging system 300 performs the 2D rhexis registration and visualization method using the monoscopic image data.

Generally, the method for registering and visualizing the rhexis edge includes steps that are performed during the capsulorhexis phase and steps that are performed during the phacoemulsification phase (which may also be performed during the IOL implantation and alignment phase).

FIG. 4 depicts a data flow diagram 400 that illustrates the steps of the method for registering and visualizing a rhexis edge that are performed during the capsulorhexis phase, in accordance with embodiments of the present disclosure. FIGS. 5A, 5B depict data flow diagrams 500, 502 (respectively) that illustrate the steps of the method for registering and visualizing a rhexis edge that are performed during the phacoemulsification phase, in accordance with embodiments of the present disclosure.

### 2D Rhexis Registration and Visualization Method

In certain embodiments, the method for registering and visualizing a rhexis edge (2D rhexis registration and visualization) includes detection of the rhexis edge during the capsulorhexis phase and registration and visualization of the rhexis edge during the phacoemulsification phase. In some embodiments, registration of the rhexis edge includes procrustes alignment on contour points. In other embodiments, the registration of the rhexis edge includes random sample consensus (RANSAC) robust affine estimation.

With respect to FIG. 4, at block 410, the ophthalmic surgical image processing system 280 receives the video stream from the stereoscopic camera 284 or the control system 340 receives the video stream from the video camera 330. The video stream is acquired during the capsulorhexis phase and includes a first sequence of video frames. Each video frame includes first image data 420 of the eye 110. The first image data 420 are presented on the touch screen displays 256, 257, and/or 286. The sclera 112, the limbus 116, the iris 120, and/or the pupil 122 (or any combinations thereof) are identified in the first image data 420.

The functionality described at blocks 432, 433, 434, 435, 436, 437, and 438 is performed by the processor 292 of the ophthalmic surgical image processing system 280 or the processor 292 of the control system 340 in real time before the first image data 420 in the next video frame is received. As discussed herein, “real-time” processing refers to performing the functionality described at blocks 432, 433, 434, 435, 436, 437, and 438 with sufficiently low latency that the data are stored and the preliminary overlay 440 is generated and available substantially simultaneously with the first image data 420 in each video frame, enabling immediate use without perceptible delay to the surgeon.

In certain embodiments, the processor 292 may execute an ML model 430 to perform some or all of the functionality described at blocks 432, 433, 435, 436, and 438 (blocks 434 and 437 simply store data in the memory 294 or 254). The ML model 430 may comprise one or more models, such as a single model, a group of models, and so on. For example, although each of the blocks 432, 433, 435, 436, and 438 is described as being performed by the ML model 430, each block could be performed by the same model, a different model in a group of models, and so on, referred to herein as the ML model 430.

At block 432, the ML model 430 processes the first image data 420 to detect the pupil mask and the rhexis edge of the eye 110.

In certain embodiments, the ML model 430 detects a segmentation mask, such as a binary segmentation mask, that identifies the pupil 122 based on the first image data 420. The segmentation mask distinguishes the pupil 122 from other anatomical structures in the first image data 420, such as the inner boundary of the iris 120, and so on. The ML model 430 detects the rhexis edge by generating a rhexis edge polyline.

In certain embodiments, the ML model 430 may include a deep learning segmentation model that includes one or more neural networks that is trained to detect the pupil mask using a supervised learning approach based on labeled ophthalmic image data. For example, a convolutional neural network (CNN) having a U-Net architecture may be trained on a dataset of surgical or diagnostic images of eyes in which the pupil has been annotated, such as through pixel-level segmentation masks identifying the pupil boundary. During training, each input image is provided to the model along with a corresponding ground truth mask, and the model learns to generate a predicted segmentation mask that distinguishes the pupil from surrounding anatomical structures.

The training process may include optimizing model parameters using a loss function that quantifies differences between the predicted masks and the ground truth masks, such as a cross-entropy loss, Dice loss, or a combination thereof. The dataset may include images captured under varying lighting conditions, magnifications, and anatomical variations to improve generalization. Through iterative training, the model learns spatial and textural features associated with the pupil, enabling the trained model to identify and segment the pupil in new image data acquired during an ophthalmic surgical procedure.

In certain embodiments, the ML model 430 may also include a hybrid convolutional-recurrent network or a vision transformer adapted for sequential point prediction that is trained to detect the rhexis edge by generating a polyline. A specialized dataset of intraoperative video frames or images from capsulorhexis procedures is curated, captured under the dynamic conditions of the operating microscope including varying illumination, fluid turbulence, and instrument interactions. Expert ophthalmic surgeons annotate each frame by tracing the CCC edge as a closed sequence of vertices forming a precise polyline, meticulously marking the torn anterior capsule boundary while accounting for partial occlusions by instruments or viscoelastic material. This richly labeled dataset, augmented with techniques such as geometric transformations, brightness and contrast jitter, motion blur simulation, and synthetic additions of surgical tools or blood, provides the supervised training signal necessary for the ML model 430 to learn robust rhexis edge detection in the challenging microsurgical environment.

The ML model 430 processes the first image data 420 to output an ordered list of polyline coordinates representing the rhexis edge contour. Training proceeds over numerous epochs by optimizing a composite loss that penalizes vertex displacement via Euclidean and chamfer distances, enforces boundary smoothness and closure, and incorporates temporal consistency losses when processing video sequences to maintain stable tracking across frames. The ML model 430 is then validated on unseen surgical cases using metrics such as mean absolute vertex error, Hausdorff distance, and Dice-like overlap adapted for polylines.

At block 433, the ML model 430 generates a pupil contour (C_{f}) based on the pupil mask.

In certain embodiments, the ML model 430 may also include a CNN with recurrent layers or a transformer-based point regression network. A large dataset of binary pupil segmentation masks derived from eye images captured under diverse lighting, gaze directions, and physiological conditions such as dilation or constriction is first curated. Each mask is paired with its corresponding ground-truth contour represented as an ordered sequence of vertices forming a closed polyline that accurately traces the pupil boundary, often annotated by experts or generated through high-precision edge detection algorithms applied to the original images. Data augmentation techniques including random rotations, scaling, elastic deformations, and simulated noise or partial occlusions are applied to improve the model's robustness to variations commonly encountered in real-world ophthalmic imaging or surgical video.

The ML model 430 is then trained end-to-end to input the binary pupil mask and directly regress the ordered list of polyline vertex coordinates. The training process optimizes a composite loss function that combines point-wise Euclidean distance errors, chamfer distance for overall shape fidelity, curvature regularization to enforce smoothness, and a closure constraint to ensure the predicted polyline forms a valid closed contour. After multiple epochs of optimization with appropriate learning rate scheduling, the model is evaluated on a held-out test set using metrics such as mean absolute vertex error, Hausdorff distance, and Dice-like overlap adapted for contour evaluation.

At block 434, the processor 292 stores the pupil contour (C_{f}) and the rhexis edge in the memory 294 or 254.

In some embodiments, the ML model 430 also processes the first image data 420 to detect a limbus mask and generate a limbus contour (Cₗ), as described with reference to blocks 435, 436, and/or 437.

At block 435, the ML model 430 processes the first image data 420 to detect the limbus mask.

The ML model 430 detects a segmentation mask, such as a binary segmentation mask, that identifies the limbus 116 based on the first image data 420. The segmentation mask distinguishes the limbus 116 from other anatomical structures in the first image data 420, such as the outer boundary of the iris 120, the sclera 112, and so on. The ML model 430 may be trained to detect the limbus mask as described above at block 432 with respect to the pupil 122.

At block 436, the ML model 430 generates a limbus contour (Cₗ) based on the limbus mask.

The ML model 430 may be trained to generate the limbus contour (Cₗ) in a similar or comparable manner as described above at block 433 with respect to the pupil 122 but as applied to the limbus 116.

At block 437, the processor 292 stores the limbus contour (Cₗ) in the memory 294 or 254 with the pupil contour (C_{f}) and/or the rhexis edge.

In certain embodiments, the ML model 430 generates a preliminary overlay identifying the pupil contour (C_{f}), the rhexis edge, and/or the limbus contour (Cₗ).

At block 438, the ML model 430 generates a preliminary overlay 440 that includes the pupil contour 450 and/or the rhexis edge 460. The pupil contour 450 is indicated by a graphical element (such as a semi-transparent color-coded contour line), and the rhexis edge 460 is indicated by another graphical element (such as a semi-transparent, color-coded polyline). In some embodiments, the preliminary overlay 440 also identifies the limbus contour 470.

The processor 292 then presents the preliminary overlay 440 with the first image data 420 on a display of the optical imaging system, such as the touch screen display 286 (and/or the touch screen displays 256, 257) of the optical imaging system 200 or the touch screen displays 256, 257 of the optical imaging system 300. In some embodiments, the processor 292 presents the preliminary overlay 440 with the first image data 420 on one or more displays within the stereoscopic microscope 240 which is visible through the binoculars, or on a display within the monoscopic microscope 320 which is visible through the eyepieces.

The pupil contour 450, the rhexis edge 460, and/or the limbus contour 470 are determined in real-time based on the first image data 420 within each video frame of the video stream. Accordingly, as the eye 110 moves during the surgical procedure, the pupil contour 450, the rhexis edge 460, and/or the limbus contour 470 follow the movement of the eye 110 in the video stream. Advantageously, the eye 110 does not need to be centered in each video frame in order to calculate the pupil contour 450 and the rhexis edge 460 (and the limbus contour 470) in each video frame.

In certain embodiments, the ML model 430 uniformly resamples each pupil contour (C_{f}) and/or each limbus contour (Cₗ) to N points before further processing.

With respect to FIG. 5A, at block 510, the ophthalmic surgical image processing system 280 receives the video stream from the stereoscopic camera 284 or the control system 340 receives the video stream from the video camera 330. The video stream is acquired during the phacoemulsification phase and includes a second sequence of video frames. Each video frame includes second image data 520 of the eye 110. The second image data 520 are presented on the touch screen displays 256, 257, and/or 286. The sclera 112, the limbus 116, the iris 120, and/or the pupil 122 (or any combinations thereof) are identified in the second image data 520.

The functionality described at blocks 532, 533, 534, 535, 536, 537, 538, and 539 is performed by the processor 292 of the ophthalmic surgical image processing system 280 or the processor 292 of the control system 340 in real time before the second image data 520 in the next video frame is received. As discussed herein, “real-time” processing refers to performing the functionality described at blocks 532, 533, 534, 535, 536, 537, 538, and 539 with sufficiently low latency that the overlay 540 is generated and available substantially simultaneously with the second image data 520 in each video frame, enabling immediate use without perceptible delay to the surgeon.

In certain embodiments, the processor 292 may execute an ML model 430 to perform some or all of the functionality described at blocks 532, 533, 534, 535, 536, 537, 538, and 539. As discussed above, the ML model 430 may comprise one or more models, such as a single model, a group of models, and so on. For example, although each of the blocks 532, 533, 534, 535, 536, 537, 538, and 539 is described as being performed by the ML model 430, each block could be performed by the same model, a different model in a group of models, and so on, referred to herein as the ML model 430.

At block 532, the ML model 430 processes the second image data 520 to detect the pupil mask of the eye 110, as discussed above with respect to block 432.

At block 533, the ML model 430 generates a second pupil contour (C_{g}) based on the pupil mask, as discussed above with respect to block 433.

In some embodiments, the ML model 430 also processes the second image data 520 to detect a limbus mask and generate a second limbus contour (Cₘ). In certain situations, the pupil mask of the eye 110 may not be detectable or the pupil mask may not support the generation of the second pupil contour (C_{g}). In these situations, the limbus may be used instead of the pupil to calculate the spatial transform. For example, the limbus contour (Cₗ) and the second limbus contour (Cₘ) may be used instead of the pupil contour (C_{f}) and the second pupil contour (C_{g}) in the calculations described above. In other embodiments discussed below, the calculation and application of the spatial transformation may be based on the limbus contours (Cₗ) and (Cₘ) as well as the pupil contours (C_{f}) and (Cp).

Referring to FIG. 5B, at block 535, the ML model 430 processes the second image data 520 to detect the second limbus mask, as discussed above with respect to block 435.

At block 536, the ML model 430 generates a second limbus contour (Cₘ) based on the limbus mask, as discussed above with respect to block 436.

In certain embodiments, the ML model 430 uniformly resamples each pupil contour (C_{g}) (and/or each limbus contour (Cₘ)) to N points before further processing.

Referring back to FIG. 5A, at block 534 the ML model 430 selects, from the memory 294 or 254, the pupil contour (C_{f}) (e.g., the pupil contour (C_{f}) from the first video stream as described above with reference to FIG. 4) that matches the second pupil contour (C_{g}). In certain embodiments, the ML model 430 selects the pupil contour (C_{f}) that closely matches the second pupil contour (C_{g}) based on a dimension of the pupil contour (C_{f}) and the second pupil contour (C_{g}), such as the contour perimeter or the contour area. In some embodiments, there is a recognition that the second pupil contour (C_{g}) may be in a different location in the second image data 520, e.g., due to eye movement.

At block 537, the ML model 430 calculates a spatial transformation based on the pupil contour (C_{f}) and the second pupil contour (C_{g}).

In certain embodiments, the ML model 430 calculates the spatial transformation by performing Procrustes alignment on the pupil contour (C_{f}) and the second pupil contour (C_{g}). For example, the ML model 430 may use the “scipy.spatial.procrustes” function between the pupil contour (C_{f}) and the second pupil contour (C_{g}) to produce aligned points and disparity to recover the composite scale s, rotation R, and translation t of the Procrustes alignment spatial transformation described in the next paragraph.

Procrustes alignment on contour points involves contour extraction, uniform sampling, Procrustes analysis, and a Procrustes alignment spatial transformation. The contour extraction includes retrieving the largest connected boundary of the pupil mask (or limbus mask) in both the capsulorhexis video frames (the source images) and the phacoemulsification video frames (the target images). Uniform sampling includes uniformly sampling N boundary points from each contour to form two point sets A, B∈R^{N*2}. The Procrustes analysis includes applying the SciPy function “spatial.procrustes”, which centers, scales, and rotates B (e.g., the point set from the second pupil contour (C_{g})) to best match A (e.g., the point set from the pupil contour (C_{f})) by minimizing the sum of squared distances. The resulting aligned coordinates of B provide the composite scale s, rotation R, and translation t, and represent the Procrustes alignment spatial transformation.

In other embodiments, the ML model 430 calculates the spatial transformation by estimating an affine transformation on the pupil contour (C_{f}) and the second pupil contour (C_{g}) using a Random Sample Consensus (RANSAC) procedure. For example, the ML model 430 may use the OpenCV function “cv2.estimateAffinePartial2D” on the two contours with RANSAC for outlier robustness.

RANSAC robust affine estimation involves contour extraction and sampling similar to above, robust 4-DOF affinity. The robust 4-DOF affinity includes using the OpenCV function “cv2.estimateAffinePartial2D (A, B, method=cv2.RANSAC)” to compute an affine matrix and rejecting boundary outliers via RANSAC. The affine matrix M represents the affine spatial transformation and is provided as Equation 1, where tₓ is a translation in X, t_{y} is a translation in Y, θ is a rotation angle, and s is a uniform scaling factor:

$$M = [ \begin{matrix}s cos θ & - s sin θ & {t}_{x} \\ s sin θ & s cos θ & {t}_{y}\end{matrix} ] \qquad Eq . l$$

At block 538, the ML model 430 applies the spatial transformation to the rhexis edge stored with the selected pupil contour (C_{f}) to generate a transformed rhexis edge.

In certain embodiments, the Procrustes alignment spatial transformation is applied to the rhexis edge to generate the transformed rhexis edge. In other embodiments, the affine spatial transform is applied to the rhexis edge to generate the transformed rhexis edge, which may include applying the OpenCV functions “cv2.warpAffine” or “cv2.transform with M” to the rhexis edge.

In some embodiments, the calculation and application of the spatial transformation may be based on the limbus contours (Cₗ) and (Cₘ) instead of or in addition to the pupil contours (C_{f}) and (Cp). For example, the ML model 430 may select, from the memory 294 or 254, the limbus contour (Cₗ) that matches the second limbus contour (Cₘ), calculate the spatial transformation based on the limbus contour (Cₗ) and the second limbus contour (Cₘ), and apply the spatial transformation to the rhexis edge associated with the selected limbus contour (Cₗ) to generate the transformed rhexis edge.

At block 539, the ML model 430 generates an overlay 540 that includes the pupil contour 550 and the transformed rhexis edge 560 (and the second limbus contour (Cₘ)). The pupil contour 550 is indicated by a graphical element (such as a semi-transparent color-coded contour line), and the rhexis edge 560 is indicated by another graphical element (such as a semi-transparent color-coded polyline). In some embodiments, the overlay 540 also includes the limbus contour 570 which is indicated by another graphical element (such as a semi-transparent color-coded polyline). A dashed arrow with a duplication of the overlay 540 outside of the second image data 520 of the eye 110 is provided for ease of visibility and understanding.

In some embodiments, the overlay 540 and user interface (UI) or user experience (UX) design provides semi-transparency, color and line style, handle buttons, and voice commands. The transformed rhexis edge 560 may be rendered as a semi-transparency contour at ~50% opacity to avoid obscuring critical microstructures (e.g., red reflex). For the color, line style, and/or weight; surgeon-adjustable palettes (green/yellow/red) and dashed or dotted line patterns and/or weights may be used to distinguish the transformed rhexis edge 560 from other computer-generated graphics. For example, the ophthalmic microscope handle buttons may be moved up/down to adjust opacity in 10% increments, moved left/right to cycle color schemes and line styles, center-pressed to re-anchor the overlay if misaligned. Voice commands may be integrated or voice-recognition modules may provide hands-free commands, such as “hide rhexis,” “show 3D mode,” “increase contrast”, and so on.

The processor 292 then presents the overlay 540 with the image data 520 on a display of the optical imaging system, such as the touch screen display 286 (and/or the touch screen displays 256, 257) of the optical imaging system 200 or the touch screen displays 256, 257 of the optical imaging system 300. In some embodiments, the processor 292 presents the overlay 540 with the image data 520 on a display within the stereoscopic microscope 240 which is visible through the binoculars, or on a display within the monoscopic microscope 320 which is visible through the eyepieces. The pupil contour 550 and the transformed rhexis edge 560 (and the limbus contour 570) are determined in real-time based on the image data 520 within each video frame of the video stream.

### 2D/3D Rhexis Registration and Visualization Method

2D/3D rhexis edge registration and visualization uses the stereoscopic camera 284 of the optical imaging system 200 to provide stereoscopic image data with two synchronized views (left and right) from which depth (Z) can be recovered. With calibrated stereo image data and a known actual limbus width from preoperative biometry, a 3D reconstruction of key anatomical landmarks can be obtained. The 2D/3D registration and visualization also involves stereo reconstruction of the video frames in the capsulorhexis phase, normalization in 3D, averaging over a sequence, 3D registration in the phacoemulsification phase, and projection back to 2D.

The stereo reconstruction of the video frames in the capsulorhexis phase includes running the ML model 430, computing disparity, and reconstructing 3D coordinates. The ML model 430 (or a stereo matching process) is run on the left and right images to obtain the pupil mask and rhexis edge. The disparity (e.g., difference in x-coordinates or in y-coordinates) is computed for corresponding points on the pupil and limbus, and the depth Z is calculated using Equation 2:

$$Z = \frac{f * B}{d} \qquad Eq . 2$$

Where f is the focal length, B is the baseline between cameras, and d is the disparity. The 3D coordinates are reconstructed for each point on the rhexis edge (or polyline), i.e., (X, Y, Z), and the known limbus width is used to calibrate the scale and resolve any ambiguity.

The normalization in 3D includes extracting robust features from the pupil by computing its 3D center (e.g., by combining 2D detection with a fixed or computed depth) and its effective radius. Then, each point P_{3D} in the 3D rhexis edge is converted into relative coordinates Pᵣₑₗ using Equation 3:

$${P}_{r e l} = \frac{{P}_{3 D} - {C}_{3 D}}{r} \qquad Eq . 3$$

For the relative coordinates, C_{3D} is the 3D pupil center and r is the scale derived from the pupil (or limbus) dimensions.

Averaging over the first sequence of video frames includes resampling and averaging the 3D polylines over all capsulorhexis frames, yielding an average relative 3D rhexis edge.

In certain embodiments, a limbus contour (Cₗ) is generated based on the first image data 420, a disparity for corresponding points on the pupil contour (C_{f}) and the limbus contour (Cₗ) is calculated, a depth is calculated based at least in part on the disparity, three-dimensional (3D) rhexis edge coordinates are generated based on the rhexis edge and the depth, a 3D pupil center PC and an effective radius are determined, normalized 3D rhexis edge coordinates are generated based on the 3D pupil center PC_{f} and the effective radius, and an average 3D rhexis edge is generated based on the normalized 3D rhexis edge coordinates in each video frame in the first sequence of video frames.

For the 3D registration in the phacoemulsification phase, once a set of 3D points (e.g., from the rhexis edge) is reconstructed in the capsulorhexis phase, these points are registered (i.e. aligned) to a corresponding set of 3D landmarks extracted the second image data 520 in the phacoemulsification phase. The registration can be formulated as finding a rigid transformation T (or similarity transformation) that minimizes the distance between corresponding points. The rigid transformation T includes rotation and translation.

The 3D registration involves computing 3D features, determining the rigid transformation T, which includes rotation and translation, and applying T. The 3D features (pupil center, and so on) are computed from the second image data 520. The rigid transformation T aligns the reference 3D landmarks from the capsulorhexis phase with those in the phacoemulsification phase. The transformation T(q) is calculated using Procrustes analysis as given by Equation 4:

T(q)=R(q)+t Eq. 4

Where R(q) is a rotation matrix (3×3), q is a 3D point from the reference (capsulorhexis) phase, and t is a translation vector in R³. Then, T(q) is applied to the averaged relative 3D rhexis edge. The parameters R(q) and t can be estimated by CNNs or deterministic methods such as Procrustes analysis or the Iterative Closest Point (ICP) method using a set of corresponding landmarks (e.g., the pupil center and several points along the limbus). With the accurate depth recovered using f and B and scaled by the limbus width, the transformation T(q) is computed in true physical (mm) units.

The projection back to 2D involves using the intrinsic projection matrix K of the stereoscopic camera 284 to project the transformed 3D points onto the 2D image plane of the phacoemulsification frame using Equation 5:

$${P}_{2 D} = ( \begin{matrix}\frac{{f}_{x} X}{Z} + {c}_{x} \\ \frac{{f}_{y} Y}{Z} + {c}_{y}\end{matrix} ) \qquad Eq . 5$$

In Eq. 5, P_{2D} is the projected point, X, Y, and Z are the coordinates of the transformed 3D point, fₓ and f_{y} are the camera focal lengths in pixels (along x-axis and y-axis), and cₓ and c_{y} are the coordinates of the camera principal point (such as the optical center) in pixels. The intrinsic projection matrix K (also referred to as the intrinsic camera matrix or the calibration matrix) is a 3×3 matrix that models the internal geometry of the stereoscopic camera 284 and may be obtained during the initial calibration of the camera. The intrinsic projection matrix K provides the camera focal lengths fₓ and f_{y}, the camera principal point coordinates cₓ, and c_{y}, and a skew parameter s. The projection provides the 2D overlay 540 of the transformed rhexis edge 560 on the image data 520 of the phacoemulsification frame, as depicted in FIG. 5A.

For both 2D and 2D+3D methods, while pupil and limbus masks provide robust landmarks, other anatomical features (e.g., sclera contours and preoperative OCT data) can further improve registration.

The edge of the sclera is not subject to the rapid changes of the pupil. Incorporating scleral landmarks (for example, the “white-to-white” measurement) adds another robust constraint. Detecting and extracting the scleral contour from the image allows several corresponding points to be included in the registration matching. These points help fix the scale and orientation more precisely. In some cases, the white-to-white (WTW) measurement can be supplemented, normalized, etc. to account for variations in WTW uniformity, eye maladies, natural or injury-induced nonuniformity in iris shape, etc.

The preoperative OCT data provides volumetric, high-resolution 3D images of the anterior segment, including the cornea 114, limbus 116, and anterior portion of the lens capsule 126. Using segmentation techniques on the OCT volume, the 3D geometry of the lens capsule 126, limbus 116, and cornea 114 may be extracted. During the ophthalmic surgery, the stereo reconstructed points (from the 2D stereo camera system) are registered to the preoperative OCT model. This fusion of intraoperative and preoperative data can be achieved using deformable or rigid registration (e.g., using iterative closest point (ICP) with regularization). The known limbus width from biometry helps ensure that the scales of the OCT volume and the stereo reconstruction are matched or are otherwise consistent.

In certain embodiments, a second limbus contour (Cₘ) is generated based on the second image data 520, a 3D pupil center PC_{g} is determined based on the second image data 520, the spatial transformation is calculated based on the 3D pupil centers PC_{f} and PC_{g} and the limbus contour (Cₗ) and the second limbus contour (Cₘ), the spatial transformation is applied to the average 3D rhexis edge to generate a transformed 3D rhexis edge, the transformed 3D rhexis edge is projected onto a two-dimensional plane, and the overlay identifying the transformed 3D rhexis edge is generated.

In certain embodiments, the surgical procedure may include pre-operation setup, the capsulorhexis phase, the phacoemulsification phase, and the IOL implantation and alignment phase. For the pre-operation setup, system intrinsics/extrinsics may be calibrated with a factory calibration phantom to configure and align the camera, and a patient-specific capsulorhexis reference model may be loaded. For example, during the calibration of the camera, the camera views a perfectly measured test object (the phantom) and the system adjusts the camera's internal settings (intrinsics) and physical alignment (extrinsics) to ensure optimum alignment and precision. During the capsulorhexis phase, the pupil contour is generated and the rhexis edge is detected and stored in memory. During the phacoemulsification phase, the transformed rhexis edge and pupil contour are generated and displayed in an overlay. When any portion of the visible rhexis edge is obscured, the transformed rhexis edge in the overlay provides continuous guidance. During the IOL implantation and alignment phase, the transformed rhexis edge and pupil contour are also generated and displayed in the overlay to facilitate IOL centration, reducing reliance on manual marking. For example, manual marking may not be sufficiently reliable.

Advantageously, the 2D and 2D+3D methods provide various benefits to the surgeon, such as continuous guidance, ergonomic efficiency, as well as teaching and collaboration. The continuous guidance might be provided by maintained visual reference of a transformed rhexis edge even under dense cataract opacity, reducing manual guesswork and potential capsule tears. The ergonomic efficiency might be provided by a heads-up display that minimizes head tilts and ocular refocusing, improving surgeon comfort and reducing musculoskeletal strain. The teaching and collaboration might be provided by external monitors with augmented reality (AR) overlays which may enhance trainee understanding and team situational awareness, fostering operating room education. For example, a trainee may be able to more clearly see the direction and orientation of tearing by which a teaching surgeon is demonstrating a capsulorhexis. Additionally, the surgeon can work on their own or others surgical technique by focusing on precise steps, providing a benchmark based on a curated selection of reference cases, and/or training other surgeons in a more engaging way.

Certain embodiments of the present disclosure also provide an ML models 430 that is configured to identify and segment anatomical and procedural features within each frame, including but not limited to the limbus, the pupil, the sclera, the rhexis edge, Purkinje reflections, surgical instruments, and/or intraocular implants. The segmented features are represented as structured data outputs that can be further analyzed and utilized to generate intraoperative guidance.

In some embodiments, the optical imaging systems 200, 300 further integrate pre-operative measurement data, including biometric parameters associated with the patient's eye, such as limbus dimensions and estimated lens capsule geometry. The integration of pre-operative data with intraoperative imaging enables the optical imaging systems 200, 300 to generate patient-specific reference models that improve the accuracy and contextual relevance of the analysis. The optical imaging systems 200, 300 compute spatial parameters from each frame of the video stream, including but not limited to feature centers and centroids, relative positioning, diameters, angular relationships, and alignment metrics. These spatial parameters are derived in a coordinate system associated with the surgical field and may be continuously updated to reflect changes during the procedure. Furthermore, these spatial parameters may be checked against and modified to more closely align with the pre-operative data.

In addition to spatial analysis, the optical imaging systems 200, 300 are configured to compute temporal metrics based on sequences of frames, including but not limited to instrument motion trajectories, velocities, accelerations, stability measures, and variance over time. These temporal metrics enable characterization of surgical maneuvers and provide a basis for assessing procedural consistency and precision. In certain embodiments, the system generates predictive or corrective guidance based on deviations from predefined or learned target parameters. Such guidance may be presented in real time via overlays, visual indicators, or other user interface elements integrated with the touch screen displays 256, 257, and/or 286.

The optical imaging systems 200, 300 provide a framework that supports a plurality of intraoperative use cases that extend beyond simple visualization, including automated guidance for forming a capsulorhexis of a desired size and centration, monitoring and coaching of instrument movement, and registration of anatomical features for subsequent procedural phases, such as the phacoemulsification phase. Additional embodiments include dynamic modeling of the lens capsule for intraocular lens placement, monitoring of pupil behavior during surgery, and generation of quantitative metrics indicative of surgical skill or performance. By combining real-time image analysis with patient-specific data and procedural modeling, the optical imaging systems 200, 300 improve surgical reproducibility and enhance clinical outcomes while reducing variability associated with manual techniques.

In certain embodiments, for each video frame, the ML model 430 detects a set of anatomical and procedural features. These features may include, but are not limited to, the limbus, pupil, sclera, and Purkinje masks (where P1-P4 may represent each of four respective Purkinje images generated by reflections from the outer surface of the cornea, the inner surface of the cornea, the outer surface of the lens, and the inner surface of the lens, respectively), each of which provides optical and geometric reference information. In certain embodiments, additional detected elements may include a rhexis edge polyline, as well as masks corresponding to surgical instruments and implanted devices. In some embodiments, an optional blood-vessel mask may also be identified to provide further anatomical context and to assist in stabilization or registration across video frames.

In certain embodiments, the ML model 430 may determine a variety of clinically and procedurally relevant parameters based on the detected features within each video frame. For example, the limbus (corneal) center may be determined as the centroid of the limbus mask, while the pupil center may be defined as the centroid of the pupil mask. A visual-axis point may be derived as the intersection of the pupil center and the optical axis, which may be obtained through geometric calibration. Additional parameters may include rhexis centration and diameter, which may be calculated by fitting a best-circle to the rhexis edge polyline, as well as gaze angle, defined as the angle between the visual-axis and the microscope axis.

In certain embodiments, additional derived parameters may provide information relating to ocular alignment and surgical precision. For example, angle k and chord u may be calculated as ocular angle metrics derived from the relative positions of the limbus and pupillary centers. In another example, instrument tip trajectory may be obtained by tracking instrument masks across sequential video frames, thereby enabling analysis of motion and control. In a further example, IOL tilt and decentration may be inferred from asymmetries observed in higher-order Purkinje reflections, including the 3^{rd} (P3) and 4^{th} (P4) reflections, thereby providing a measure of implant positioning accuracy.

In certain embodiments, parameters may additionally be evaluated across multiple video frames through temporal tracking. By linking per-frame parameters, dynamic characteristics such as velocities and accelerations of instrument tips and edges may be calculated, thereby providing insight into surgical motion patterns. Changes in rhexis geometry may be quantified, including Δθ (rhexis rotation) and dD/dt (rhexis expansion rate), which reflect rotational and radial changes over time. Statistical measures such as σ (Standard Deviations) may be computed for parameters including centration, radius, and pupil stability to assess consistency and variability.

In certain embodiments, additional temporal metrics may include ΔZ (axial shifts), which may indicate lens bag movement and may be associated with zonular laxity. To ensure robustness in parameter estimation, particularly in the presence of occlusions such as viscoelastic bubbles, time-series analysis techniques may be applied. Such techniques may include, in some embodiments, Kalman filtering and smoothing splines, both of which may reduce noise and maintain continuity in tracked signals. Accordingly, in certain embodiments, reliable parameter tracking may be achieved even under challenging intraoperative conditions.

In certain embodiments, the optical imaging systems 200, 300 provide real-time guidance that assists the formation of the capsulorhexis during the capsulorhexis phase by incorporating explicit geometric and predictive modeling. In certain embodiments, the optical imaging systems 200, 300 dynamically identify and track the rhexis edge within each video frame of the video stream and calculates a geometric representation of the rhexis edge using a least-squares circle fitting formulation. For example, the fitted circle may be expressed as Equation 6:

(x−x_{c})²+(y−y_{c})²=r² Eq. 6

Where x and y represent the coordinates of the rhexis edge, x and y, represent the center coordinates of the rhexis, and r represents the rhexis edge radius. The optical imaging systems 200, 300 may determine these parameters by minimizing an algebraic error term using a method such as Pratt's circle fit, thereby providing a robust estimation of the evolving capsulorhexis geometry.

In some embodiments, the optical imaging systems 200, 300 perform scale calibration to convert image-space measurements into physical units by leveraging anatomical reference features and preoperative data. For example, a pixel-to-millimeter conversion factor S may be defined as using Equation 7:

$$S = \frac{{D}_{limbus}^{m m}}{{D}_{limbus}^{px}} \qquad Eq . 7$$

Where

$${D}_{limbus}^{m m}$$

is a known limbus diameter from preoperative biometry and

$${D}_{limbus}^{p x}$$

is the measured limbus diameter in pixels within the video frame. The conversion factor S enables conversion of the fitted radius r into a physical radius rₘₘ, such as rₘₘ=S·r, thereby allowing comparison against a target capsulorhexis diameter range, such as 5.0-5.5 mm. The use of patient-specific calibration improves the accuracy and clinical relevance of the guidance system.

The system is further configured to generate a predictive model of capsulorhexis evolution using temporal derivatives of the fitted parameters. In certain embodiments, the angular position θ(t) of the rhexis edge and its rate of change may be modeled using a kinematic formulation as provided by Equation 8:

$${θ}_{1} = {θ}_{0} + {ω}_{0} Δ t + \frac{1}{2} {{α}_{0} ( Δ t )}^{2} \qquad Eq . 8$$

In Eq. 8, θ₁ represents the new angular position at time interval Δt, θ₀ represents the angular position, ω₀ represents the angular velocity, and α₀ represents angular acceleration. Similarly, radial position r(t) may be expressed as Equation 9:

r₁=r₀+v₀Δt Eq. 9

In Eq. 9, r₁ represents the new radial position at time interval Δt, r₀ represents the radial position, and v₀ represents radial velocity. Equation 9 enables prediction of the future capsulorhexis boundary over a short time horizon. These predictive calculations enable the optical imaging systems 200, 300 to generate a forecasted circle representing the expected edge position within a defined interval, such as 1-2 seconds ahead.

The optical imaging systems 200, 300 may be configured to render a dynamic graphical overlay corresponding to a target circle. The target circle may be defined by Equation 10:

$${( x - {x}_{c} )}^{2} + {( y - {y}_{c} )}^{2} = {r}_{t a r g e t}^{2} \qquad Eq . 10$$

In Eq. 10, x and y represent the coordinates of the rhexis edge, x_{c} and y_{c} represent the center coordinates of the target circle, and r_{target} represents the radius of the target circle. The optical imaging systems 200, 300 may also be configured to render a dynamic graphical overlay corresponding to a predictive circle by extrapolating the edge curvature and angular velocity of the target circle via a short-term parametric model (e.g., constant-acceleration), yielding a predicted circle 1-2 seconds ahead. Both the target circle and the predictive circle may be scaled to the diameter of the limbus using the limbus contour Cₗ.

Visual attributes of the overlays may be modulated based on deviation metrics, such as Δr=|rₘₘ−r_{target}|, where color transitions (e.g., green or red) correspond to predefined tolerance thresholds. Additional notification conditions may be defined using inequalities, such as triggering a visual or auditory alert when Δr>0.5 mm, and confirming acceptable performance when Δr≤0.1 mm. By integrating explicit geometric equations, calibrated scaling, and/or predictive kinematic modeling, the system is able to provide precise and anticipatory guidance that enhances surgical accuracy and consistency.

Referring to FIG. 4, the ML model 430 may include additional functionality between block 437 and block 438 to generate the target circle and the predicted circle, and block 438 may be modified to include the target and predictive circles in the preliminary overlay 440.

In certain embodiments, for each video frame in the first sequence of video frames, the ML model 430 generates the target circle based on the coordinates of the rhexis edge and the limbus contour (Cₗ) (as described above), and generates the preliminary overlay further identifying the target circle. In some embodiments, two concentric target circles may be generated to provide a guidance ring or annulus.

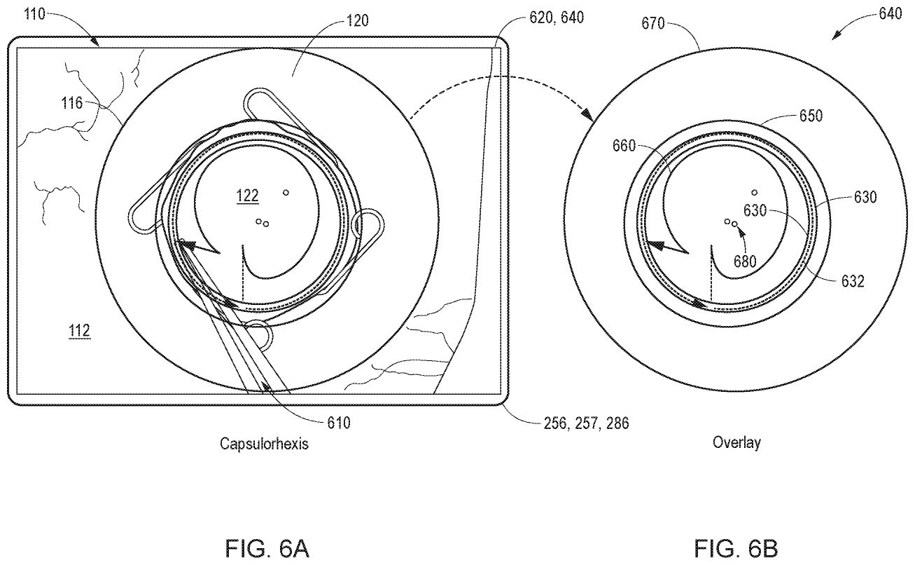

FIG. 6A depicts the image data 620 from a video frame with the preliminary overlay 640, in accordance with embodiments of the present disclosure. The image data 620 and the preliminary overlay 640 are presented on the touch screen displays 256, 257, and/or 286. The sclera 112, the limbus 116, the iris 120, and the pupil 122 of the eye 110 and a forceps 610 are identified in the image data 620.

FIG. 6B depicts the preliminary overlay 640, in accordance with embodiments of the present disclosure. The target circle 630, the predictive circle 632, the pupil contour 650, and the limbus contour 670 are depicted. The pupil contour 650 is the same as the pupil contour 450, and the limbus contour 670 is the same as the limbus contour 470. The preliminary overlay 640 also identifies the Purkinje masks 680.

Known surgical navigation tools do not provide real-time corrective feedback that reflects the surgeon's actual instrument motion. As a result, deviations from the intended trajectory may go unaddressed until after they have already impacted the procedure. Embodiments of the present disclosure address this limitation by continuously tracking the forceps tip to generate a forceps tip path during a capsulorhexis procedure, calculating deviation vectors relative to an ideal circular arc, and delivering immediate corrective guidance through visual, haptic, and audio modalities.

In certain embodiments, the optical imaging systems 200, 300 establish a closed-loop feedback mechanism that dynamically compares the surgeon's current motion against a predefined optimal trajectory. By doing so, the optical imaging systems 200, 300 enable continuous intraoperative adjustment rather than passive observation of path deviation. This approach improves precision and consistency during capsulorhexis by actively guiding the surgeon toward the desired motion profile in real time.

In certain embodiments, the ML model 430 performs tip localization by computing the centroid of the segmented forceps-tip mask for each frame of acquired imaging data. The ML model 430 may smooth this centroid using a constant-velocity Kalman filter, thereby reducing jitter and improving temporal stability of the tracked position. The resulting filtered position is denoted as Pₜᵢₚ and serves as the basis for subsequent calculations.

The ideal path for the tip may be defined by parameterizing a desired circular segment P_{ideal}(θ), which is derived from the measured rhexis center and radius. This parameterization enables the ML model 430 to represent the intended trajectory as a continuous function of angular position. The circular arc serves as a reference against which real-time deviations are quantified. Note that, at the start of the formation of the rhexis, the ideal path may be the target circle 630. As the rhexis is formed, the rhexis edge is detected, a rhexis center and radius are determined, and the ideal path is updated.

The deviation vector may be computed as ΔP=Pₜᵢₚ−P_{ideal}, representing the instantaneous positional error between the actual tip location and the ideal path. The magnitude of this deviation, ∥ΔP∥, may be used to determine the degree of corrective guidance required. Larger deviations correspond to stronger visual and sensory feedback, thereby enabling proportional correction by the surgeon.

The ML model 430 may generate the preliminary overlay by rendering the ideal circular arc as a dashed line within the surgical visualization interface. In addition, a corrective arrow may be displayed extending from the current tip position toward the nearest point on the ideal path, with the arrow length and direction determined by ΔP. This visual cue provides intuitive, real-time guidance to assist the surgeon in correcting deviations.

In some embodiments, biological properties of the capsular bag and the rate it which it tears are also included in the rendering of the corrective guidance. For example, a direct path from the current position (which may be off of the ideal path) may cause an unfavorable tear or shape to the rhexis. In these and other embodiments, a more gradual route back to the ideal path may be provided in the preliminary overlay 640. In some embodiments, clinical outcomes of different shaped rhexes may also be included as a factor in the model predicting the path back to the ideal path. For example, a sharp turn may increase the likelihood of capsular bag failure or other complications during the procedure, and the balance between the ideal size and ideal curvature of the rhexis may be balanced against a more aggressive path back to the ideal path.

The ML model 430 may highlight errors by modifying the visual characteristics of the corrective arrow based on the magnitude of deviation. For example, the arrowhead may be configured to glow yellow when ∥ΔP∥>0.5 mm, thereby signaling a threshold level of positional error. This visual emphasis ensures that significant deviations are readily noticeable without overwhelming the display.

In some embodiments, haptic and audio feedback mechanisms may be provided to reinforce corrective guidance. When the deviation persists for greater than one second, a gentle audible beep may be emitted and, in certain embodiments, a corresponding vibration may be applied to a foot-pedal interface or other component of surgical equipment. These multimodal cues ensure that the surgeon receives timely and perceivable feedback, even when visual attention is focused elsewhere within the surgical field.

Referring to FIG. 4, the ML model 430 may include additional functionality between block 437 and block 438 to generate a forceps tip path, and block 438 may be modified to include the forceps tip path in the preliminary overlay 440.

In certain embodiments, for each video frame in the first sequence of video frames, the ML model 430 generates a forceps tip mask based on the first image data, calculates a forceps tip path based on the forceps tip mask in the video frame and the forceps tip mask in previous video frames, calculates a deviation of the forceps tip mask from the target circle, generates a correction arrow based on the deviation, and generates the preliminary overlay further identifying the forceps tip path.

Referring to FIGS. 6A, 6B, the preliminary overlay 640 identifies the forceps tip path 660 after the CCC has been created.

In certain embodiments, the spatial transformation applied to the rhexis edge detected at block 432 to generate the transformed rhexis edge at block 538 may be calculated based on the limbus contours (Cₗ) generated at block 437, the second limbus contour (Cₘ) generated at block 536, and/or Purkinje reflections P1-P4 that are detected during the capsulorhexis and phacoemulsification phases.

The ML model 430 detects the Purkinje reflections P1-P4 as stable optical features, while limbus contours Cₗ, Cₘ provide geometric context. These features collectively serve as anchor points for establishing correspondence between frames.

The ML model 430 may estimate the spatial transformation using a robust fitting technique, such as RANSAC, to determine a similarity transform defined by (s,R,t). The spatial transformation is calculated by minimizing the reprojection error between matched key points across the capsulorhexis video frames and the phacoemulsification video frames. The resulting spatial transformation parameters enable accurate alignment of the stored rhexis edge with the current surgical view during the phacoemulsification phase.

The spatial transformation T encapsulates the estimated scale, rotation, and translation required to map the reference geometry into the coordinate space for the currently-displayed video frame. By applying this spatial transformation in real time, the optical imaging systems 200, 300 ensure that the transformed rhexis edge remains spatially consistent with the observed anatomy. This continuous recalibration enhances both accuracy and reliability under varying intraoperative conditions.

The ML model 430 performs edge projection by applying the spatial transform T to the stored rhexis edge, which is represented as a polyline. The transformed rhexis edge (polyline) is then rendered onto the currently-displayed video frame as a semi-opaque overlay (e.g., a blue contour or polyline), providing a clear yet non-obstructive visual guide. This overlay allows the surgeon to maintain awareness of the rhexis edge even when it is not directly visible.

In some embodiments, the optical imaging systems 200, 300 may provide a visual confirmation mechanism using an “Edge Saved” lock-in icon that appears in the touch screen displays 256, 257, and/or 286 upon successful registration. This icon serves as an indicator that the optical imaging systems 200, 300 have achieved reliable alignment between the reference video frames and the currently-displayed video frame. To reinforce awareness, the icon may be configured to pulse at regular intervals, such as every 30 seconds, thereby reminding the surgeon of the active guidance system.

In other embodiments, additional adaptive visualization may be implemented through an opacity modulation feature that responds to surgeon behavior. When the optical imaging systems 200, 300 detect that the surgeon pauses for greater than two seconds near the rhexis boundary, the opacity of the overlay may be temporarily increased. This enhancement ensures that the boundary becomes more prominent during moments of focused attention, thereby facilitating precise and informed surgical actions.

Pre-operative biometry may provide accurate measurements of lens capsule geometry, including parameters derived from OCT data (such as an A-scan) and/or WTW assessments. However, intraoperative visualization of the lens capsule remains limited due to occlusion, lighting conditions, and anatomical variability. Embodiments of the present disclosure advantageously project a three-dimensional (3D) model of the lens capsule onto the live surgical field, thereby enhancing spatial awareness and supporting precise IOL placement.

In certain embodiments, the optical imaging systems 200, 300 generate a patient-specific 3D representation of the lens capsule based on pre-operative biometric data. The patient-specific lens capsule model is then mapped into the intraoperative image space using a homography-based projection technique as described below. The optical imaging systems 200, 300 then align the patient-specific lens capsule model with real-time anatomical landmarks to provide a continuously updated visual reference that reflects both the patient's anatomy and intraoperative dynamics.

This approach enables the surgeon to visualize otherwise obscured anatomical structures and reduces the likelihood of unintended zonular stress during instrument manipulation. Furthermore, the projected patient-specific lens capsule model assists in guiding IOL positioning by providing a clear indication of the intended spatial boundaries. As a result, the optical imaging systems 200, 300 improve both safety and procedural accuracy during lens implantation.

In certain embodiments, the ML model 430 performs the three-dimensional model projection by constructing a circular cross-sectional representation of the lens capsule using pre-operative biometric inputs. This model is defined in a reference coordinate system and subsequently projected into the image plane. The projection is achieved by computing a homography matrix H, which is derived from correspondences between identifiable corneal landmarks in the currently-displayed video frame and corresponding points on the model.

The homography H defines the mapping between the model space and the image plane, enabling accurate alignment of the projected geometry with the observed anatomy. This transformation accounts for perspective distortion and ensures that the projected lens capsule boundary conforms to the visual context of the surgical field. By leveraging stable anatomical features, the system maintains consistent registration throughout the procedure.

The ML model 430 adjusts the scale dynamically to compensate for intraoperative drift and minor changes in imaging conditions. In each currently-displayed video frame, the ML model 430 performs limbus segmentation to refine the estimation of H, thereby updating the scale and alignment of the projected model. This continuous recalibration ensures that the overlay remains accurate and responsive to real-time variations.

In certain embodiments, the lens capsule boundary is rendered as a thin circular overlay within the surgical view, providing a clear and intuitive representation of the anatomical structure. When the optical imaging systems 200, 300 detect that the region is out of focus, the circle may be displayed as a dashed line to indicate reduced confidence or visibility. This visual distinction helps the surgeon interpret the reliability of the overlay at any given moment. Additionally or alternatively, the weight of the line depicting the circle may be thickened to show the projection of the span of locations where the rhexis could be located.

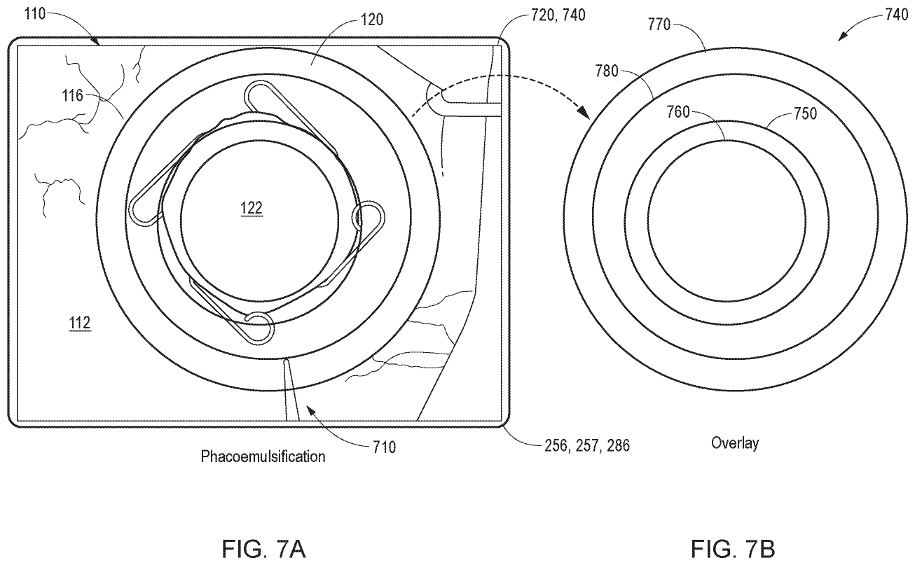

FIG. 7A depicts the image data 720 from a video frame with the overlay 740, in accordance with embodiments of the present disclosure. The image data 720 and the overlay 740 are presented on the touch screen displays 256, 257, and/or 286. The sclera 112, the limbus 116, the iris 120, and the pupil 122 of the eye 110 and a phacoemulsification instrument 710 are identified in the image data 720.

FIG. 7B depicts the overlay 740, in accordance with embodiments of the present disclosure. The pupil contour 750, the transformed rhexis edge 760, the limbus contour 770, and the lens capsule boundary 780 are depicted. The pupil contour 750 is the same as the pupil contour 550, the transformed rhexis edge 760 is the same as the transformed rhexis edge 560, the limbus contour 770 is the same as the limbus contour 570.

In some embodiments, the optical imaging systems 200, 300 may provide a proximity alert mechanism to enhance procedural safety during instrument manipulation. For example, when any instrument tip approaches within less than 0.2 mm of the projected lens capsule boundary, the circular overlay of the lens capsule boundary 780 begins to flash. This flashing behavior serves as an immediate visual warning of potential risk.

In addition to or alternatively to visual cues, an auditory notification may be generated (e.g., in the form of a low-pitched tone) when the proximity threshold is exceeded. This multimodal feedback ensures that the surgeon is promptly alerted even when visual attention is directed elsewhere. Together or separately, these notification mechanisms provide real-time guidance that supports careful and controlled interaction with the lens capsule.

Pupillary instability may be indicative of zonular laxity, iris prolapse, intraocular pressure fluctuation, or other surgical conditions that are often identified only after meaningful progression has occurred. To facilitate earlier intraoperative detection of iris-related or zonular-related abnormalities, embodiments of the present disclosure advantageously track the rhexis edge at multiple phases of the cataract surgery, including the capsulorhexis phase, the phacoemulsification phase, and the IOL implantation and alignment phase. By comparing rhexis and/or lens capsule geometry and positional relationships over time, the optical imaging systems 200, 300 enable detection of significant shape distortion or positional displacement, which may indicate zonular weakness and justify notifying the surgeon to consider preventive measures such as implantation of a capsular tension ring.

In certain embodiments, the optical imaging systems 200, 300 perform real-time analytics to determine multiple stability metrics associated with pupil behavior and rhexis-pupil relationships, thereby providing quantitative insight into intraoperative dynamics. These metrics may include rotation Δθ, dilation rate dD/dt, centroid variance GP, and ratio R_PL, each of which captures a distinct aspect of structural or functional stability. The computed values may be presented in the UI and linked to threshold-based alert conditions, enabling continuous monitoring and timely feedback to the surgeon.

The optical imaging systems 200, 300 may perform metric computation on a per-frame basis and over a sliding window of recent frames to capture both instantaneous changes and short-term trends. In certain embodiments, Δθ represents the change in angle between the pupil center and the rhexis center, while dD/dt is computed as a finite difference of pupil diameter over time to estimate dilation or constriction rate. The metric σP is defined as the standard deviation of the centroid position over the last N frames, and R_PL represents the ratio of rhexis radius to pupil radius, thereby normalizing geometric relationships across procedural stages.

The preliminary overlays 440, 640 and the overlays 540, 740 may include one or more on-screen widgets to display the computed metrics in real time, including numeric values, spark-line trends, and/or traffic-light color coding for rapid interpretation. Threshold-based notifications may be generated when predefined limits are exceeded, such as Δθ>5° or dD/dt>0.5 mm/s triggering a modal warning, or σP>0.2 mm invoking foot-pedal vibration. In some embodiments, multimodal feedback mechanisms, including visual and/or haptic cues, improve the likelihood that clinically significant instability will be detected and addressed promptly during the procedure.

Objective, AI-based assessment of surgical skill in cataract procedures remains limited, as known systems primarily evaluate broad procedural phases rather than fine-grained performance characteristics. Embodiments of the present disclosure advantageously address this limitation by determining detailed motion and outcome-based metrics, such as path length, smoothness, centration error, and completion time, thereby enabling a more precise and quantitative evaluation of surgical technique. These metrics may be further integrated into composite scoring frameworks, referred to as Composite Surgical Assessment Metrics (CSAMs), which are specifically tailored to capsulorhexis performance.

In addition to quantitative scoring, the optical imaging systems 200, 300 may provide an interactive replay environment that enables trainees to review their instrument trajectories in relation to an ideal reference path. This replay functionality includes deviation highlighting and contextual annotations, thereby transforming passive video review into an actionable training tool. By presenting both numerical and visual feedback, the optical imaging systems 200, 300 enhance skill acquisition and supports targeted improvement beyond what is achievable with conventional video-based review platforms.

In certain embodiments, the ML model 430 may calculate path length and smoothness directly from recorded instrument trajectories to characterize motion efficiency and control. Specifically, path length may be defined as L=Σ∥pᵢ₊₁−pᵢ∥, while smoothness may be defined as S=1/LΣ∥ . . . ∥Δ²pᵢ, thereby capturing cumulative distance and second-order motion variation, respectively. In some embodiments, the ML model 430 may calculate centration error as the root mean square distance between measured rhexis centers and the ideal limbus center, and a composite score may be generated as a weighted sum of normalized metrics.

In certain embodiments, a training overlay may be provided during replay, in which the recorded instrument path is superimposed on an ideal circular trajectory, with segments exhibiting significant deviation, defined as ∥Δp∥>0.5 mm, highlighted in red. Following completion of a surgical case, a post-case report may be automatically generated as a PDF scorecard that includes metric breakdowns, composite scores, and annotated video bookmarks for structured review. In some embodiments, in-situ contextual pop-up notifications may be generated during wet-lab training when metrics deviate from predefined benchmarks, thereby providing real-time corrective feedback and reinforcing proper technique.

As noted above, the actual rhexis edge created by the surgeon may be periodically obscured due to occlusion, lighting variation, or surgical manipulation. Embodiments of the present disclosure repeatedly detect and track the actual rhexis edge whenever it becomes visible, thereby maintaining continuity of anatomical reference. This capability ensures that the surgeon remains aware of the rhexis geometry when direct visualization is available.

In certain embodiments, the ML model 430 detects the rhexis geometry at multiple time points and constructs a temporally updated representation of its shape and position. When the rhexis edge is not visible, the ML model 430 generates an estimated rhexis edge based on previously captured data and overlays this estimate onto the currently-displayed video frame. When visualization resumes, the ML model 430 captures the current rhexis edge, compares it with the estimated representation, and dynamically updates the rhexis edge model to reflect intraoperative changes.

In addition to intraoperative guidance, the optical imaging systems 200, 300 may capture rhexis data at later stages, including after intraocular lens (IOL) implantation and alignment, and store the data for post-operative analysis. This data may be used to evaluate surgical performance and refine algorithms for optimizing IOL centration and positioning. Over time, aggregation of such data across multiple procedures may contribute to improved predictive models and enhanced visual outcomes for future patients.

The many features and advantages of the disclosure are apparent from the detailed specification, and, thus, it is intended by the appended claims to cover all such features and advantages of the disclosure which fall within the scope of the disclosure. Further, since numerous modifications and variations will readily occur to those skilled in the art, it is not desired to limit the disclosure to the exact construction and operation illustrated and described, and, accordingly, all suitable modifications and equivalents may be resorted to that fall within the scope of the disclosure.

What is claimed is:

1. An ophthalmic microscope, comprising:
a floor stand;
a microscope optical head rotatably coupled to the floor stand;
a monoscopic microscope rotatably coupled to the microscope optical head;
a video camera attached to the monoscopic microscope, the video camera configured to generate a video stream comprising a first sequence of video frames corresponding to a capsulorhexis phase and a second sequence of video frames corresponding to a phacoemulsification phase;
a display; and
a processor coupled to the video camera and the display, the processor configured to:
for each video frame in the first sequence of video frames:
detect a pupil mask and a rhexis edge based on first image data of an eye of a patient in the video frame;
generate a pupil contour C_{f} based on the pupil mask;
store the pupil contour C_{f} and the rhexis edge in a memory;
for each video frame in the second sequence of video frames:
detect a pupil mask based on second image data of the eye of the patient in the video frame;
generate a pupil contour C_{g} based on the pupil mask;
select, from the memory, the pupil contour C_{f} that matches the pupil contour C_{g};
calculate a spatial transformation based on the pupil contour C_{f} and the pupil contour C_{g};
apply the spatial transformation to the rhexis edge stored with the selected pupil contour C_{f} to generate a transformed rhexis edge;
generate an overlay identifying the transformed rhexis edge; and
present, on a display, the overlay with the second image data.

2. The ophthalmic microscope of claim 1, wherein the processor is further configured to:
for each video frame in the first sequence of video frames:
resample the pupil contour C_{f} to N points;
for each video frame in the second sequence of video frames:
resample the pupil contour C_{g} to N points; and
wherein selecting the pupil contour C_{f} that matches the pupil contour C_{g} is based on a dimension of the pupil contour C_{f} and the pupil contour C_{g}, and
wherein the dimension is a perimeter or an area.

3. The ophthalmic microscope of claim 2, wherein calculating the spatial transformation comprises performing Procrustes alignment on the pupil contour C_{f} and the pupil contour C_{g}.

4. The ophthalmic microscope of claim 2, wherein calculating the spatial transformation comprises estimating an affine transformation on the pupil contour C_{f} and the pupil contour C_{g} using a Random Sample Consensus (RANSAC) procedure.

5. The ophthalmic microscope of claim 1, wherein the processor is further configured to:
for each video frame in the first sequence of video frames:
detect a limbus mask based on the first image data;
generate a limbus contour Cₗ based on the limbus mask;
store the limbus contour Cₗ with the pupil contour C_{f} and the rhexis edge in the memory;
generate a preliminary overlay identifying the pupil contour C_{f}, the limbus contour Cₗ, and the rhexis edge; and
present, on the display, the preliminary overlay with the first image data.

6. The ophthalmic microscope of claim 5, wherein the processor is further configured to:
for each video frame in the second sequence of video frames:
generate a limbus contour Cₘ based on the second image data;
select, from the memory, the limbus contour Cₗ that matches the limbus contour Cₘ;
calculate the spatial transformation based on the limbus contour Cₗ and the limbus contour Cₘ;
apply the spatial transformation to the rhexis edge associated with the selected limbus contour Cₗ to generate the transformed rhexis edge;
generate the overlay identifying the pupil contour C_{g}, the limbus contour Cₘ, and the transformed rhexis edge; and
present, on the display, the overlay with the second image data.

7. The ophthalmic microscope of claim 6, wherein the processor is further configured to:
for each video frame in the first sequence of video frames:
generate a target circle based on the limbus contour Cₗ; and
generate the preliminary overlay further identifying the target circle.

8. The ophthalmic microscope of claim 7, wherein the processor is further configured to:
for each video frame in the first sequence of video frames:
generate a forceps tip mask based on the first image data;
calculate a forceps tip path based on the forceps tip mask in the video frame and the forceps tip mask in previous video frames;
calculate a deviation of the forceps tip mask from the target circle;
generate a correction arrow based on the deviation; and
generate the preliminary overlay further identifying the forceps tip path.

9. A computer-implemented method for visualizing a capsulorhexis edge during an ophthalmic surgical procedure, the method comprising:
receiving, from a camera attached to an ophthalmic microscope, a video stream comprising a first sequence of video frames corresponding to a capsulorhexis phase and a second sequence of video frames corresponding to a phacoemulsification phase;
for each video frame in the first sequence of video frames:
detecting a pupil mask and a rhexis edge based on first image data of an eye of a patient in the video frame;
generating a pupil contour C_{f} based on the pupil mask;
storing the pupil contour C_{f} and the rhexis edge in a memory;
for each video frame in the second sequence of video frames:
detecting a pupil mask based on second image data of the eye of the patient in the video frame;
generating a pupil contour C_{g} based on the pupil mask;
selecting, from the memory, the pupil contour C_{f} that matches the pupil contour C_{g},
calculating a spatial transformation based on the pupil contour C_{f} and the pupil contour C_{g};
applying the spatial transformation to the rhexis edge stored with the selected pupil contour C_{f} to generate a transformed rhexis edge;
generating an overlay identifying the transformed rhexis edge; and
presenting, on a display, the overlay with the second image data.

10. The method of claim 9, further comprising:
for each video frame in the first sequence of video frames:
resampling the pupil contour C_{f} to N points;
for each video frame in the second sequence of video frames:
resampling the pupil contour C_{g} to N points; and
wherein selecting the pupil contour C_{f} that matches the pupil contour C_{g} is based on a dimension of the pupil contour C_{f} and the pupil contour C_{g}, and
wherein the dimension is a perimeter or an area.

11. The method of claim 10, wherein calculating the spatial transformation comprises performing Procrustes alignment on the pupil contour C_{f} and the pupil contour C_{g}.

12. The method of claim 10, wherein calculating the spatial transformation comprises estimating an affine transformation on the pupil contour C_{f} and the pupil contour C_{g} using a Random Sample Consensus (RANSAC) procedure.

13. The method of claim 9, further comprising:
for each video frame in the first sequence of video frames:
detecting a limbus mask based on the first image data;
generating a limbus contour Cₗ based on the limbus mask;
storing the limbus contour Cₗ with the pupil contour C_{f} and the rhexis edge in the memory;
generating a preliminary overlay identifying the pupil contour C_{f}, the limbus contour Cₗ, and the rhexis edge; and
presenting, on the display, the preliminary overlay with the first image data.

14. The method of claim 13, further comprising:
for each video frame in the second sequence of video frames:
generating a limbus contour Cₘ based on the second image data;
selecting, from the memory, the limbus contour Cₗ that matches the limbus contour Cₘ;
calculating the spatial transformation based on the limbus contour Cₗ and the limbus contour Cₘ;
applying the spatial transformation to the rhexis edge associated with the selected limbus contour Cₗ to generate the transformed rhexis edge;
generating the overlay identifying the pupil contour C_{g}, the limbus contour Cₘ, and the transformed rhexis edge; and
presenting, on the display, the overlay with the second image data.

15. The method of claim 13, further comprising:
for each video frame in the first sequence of video frames:
generating a target circle based on the limbus contour Cₗ; and
generating the preliminary overlay further identifying the target circle.

16. The method of claim 15, further comprising:
for each video frame in the first sequence of video frames:
generating a forceps tip mask based on the first image data;
calculating a forceps tip path based on the forceps tip mask in the video frame and the forceps tip mask in previous video frames;
calculating a deviation of the forceps tip mask from the target circle;
generating a correction arrow based on the deviation; and
generating the preliminary overlay further identifying the forceps tip path.

17. The method of claim 9, wherein:
the first image data comprises stereoscopic image data;
the method further comprises:
for each video frame in the first sequence of video frames:
generating a limbus contour Cₗ based on the first image data;
calculating a disparity for corresponding points on the pupil contour C_{f} and the limbus contour Cₗ;
calculating a depth based at least in part on the disparity;
generating three-dimensional (3D) rhexis edge coordinates based on the rhexis edge and the depth;
determining a 3D pupil center PC_{f} and an effective radius;
generating normalized 3D rhexis edge coordinates based on the 3D pupil center PC_{f} and the effective radius; and
generating an average 3D rhexis edge based on the normalized 3D rhexis edge coordinates in each video frame in the first sequence of video frames.

18. The method of claim 17, further comprising:
the second image data comprises stereoscopic image data; and
the method further comprises:
for each video frame in the second sequence of video frames:
generating a limbus contour Cₘ based on the second image data;
determining a 3D pupil center PC_{g} based on the second image data;
calculating the spatial transformation based on the 3D pupil centers PC_{f} and PC_{g} and the limbus contours Cₗ and Cₘ;
applying the spatial transformation to the average 3D rhexis edge to generate a transformed 3D rhexis edge;
projecting the transformed 3D rhexis edge onto a two-dimensional plane; and
generating the overlay identifying the transformed 3D rhexis edge.

19. An ophthalmic system, comprising:
an ophthalmic microscope, comprising:
a microscope optical head; and
a stereoscopic microscope rotatably coupled to the microscope optical head;
an ophthalmic surgical image processing system comprising:
a stereoscopic camera attached to the stereoscopic microscope, the stereoscopic camera configured to generate a video stream comprising a first sequence of video frames corresponding to a capsulorhexis phase and a second sequence of video frames corresponding to a phacoemulsification phase;
a display; and
a processing system coupled to the stereoscopic camera and the display, the processing system including a processor configured to:
for each video frame in the first sequence of video frames:
generate a pupil contour C_{f} and a limbus contour Cₗ based on first stereoscopic image data of an eye of a patient in the video frame;
calculate a disparity for corresponding points on the pupil contour C_{f} and the limbus contour Cₗ;
calculate a depth based at least in part on the disparity;
generate three-dimensional (3D) rhexis edge coordinates based on a rhexis edge and the depth;
determine a 3D pupil center PC_{f} and an effective radius;
generate normalized 3D rhexis edge coordinates based on the 3D pupil center PC_{f} and the effective radius;
generate a preliminary overlay identifying the normalized 3D rhexis edge coordinates;
present, on a display, the preliminary overlay with the first stereoscopic image data; and
generate an average 3D rhexis edge based on the normalized 3D rhexis edge coordinates in each video frame in the first sequence of video frames.

20. The ophthalmic system of claim 19, wherein:
the processor is further configured to:
for each video frame in the second sequence of video frames:
generate a limbus contour Cₘ based on second stereoscopic image data;
determine a 3D pupil center PCg based on the second stereoscopic image data;
calculate a spatial transformation based on the 3D pupil centers PC_{f} and PC_{g} and the limbus contours Cₗ and Cₘ;
apply the spatial transformation to the average 3D rhexis edge to generate a transformed 3D rhexis edge;
project the transformed 3D rhexis edge onto a two-dimensional plane;
generate an overlay identifying the transformed 3D rhexis edge; and
present, on the display, the overlay with the second stereoscopic image data.
